SEQUENCE LISTING (10 sequences)

SEQ ID NO: 1 - DNA, 1066 bp, human
taaagttgcctttcaagctctggcctccgggcacgcgatgctccgcggcgggctgactcagggctgccttgggcctccctgccaccctcctggaaatgatgcaagtcctgactgtcacctggatccctgcagcccagcctggaatgcgtctggattaggggaaagacgagaaacgacactccaggtgttgcacggcccaccaaagcgggaagatagggcagttgctcagaccaaatactgtatctagtgcttctgctcctatcttcaatcgtggggttctttttaatgcaaagtgtcacaaggccaggaattcccatgtgtgctcagttggcccacagcatcattgtgcctaggaaactgcttcaatttatcaagtcctctgggctgggaatctcactgaattccaaacggcggaaagaggaaactttcccaacccgatgtgggtgtgacgcgagccaggggccccagggacactgtcccagagcacaccgtccccctttaacagcaactggagcttggattcgctcttatattgtacagtcctttcgaccattgccctggagcacccgcacacgcgcacgcatctccggccgcgctcacacacactcatacacacgcacgcaaacgcgtggccgccgccaggtcggcaactttgtccggcgctcccagcggcgctcggcttcctcctgtagtagttgagcgcaggccccgcctcccggccgtgttgtcaaaagggccggggtctcggattggtccagccgccgggacaacacctgctcgactccttcattcaagtgacaccagagcttccagggatatttgaggcaccatccctgccattgccgggcactcgcggcgctgctaacggcctggtcacatgctctccggagagctacgggagggcgctgggtaacctctatccgagccgcggccgcgaggaggagggaaaaggcgagcaaaaaggaagagtgggaggaggaggggaagcggcgaaggaggaagaggaggaggaggaagaggggagcacaaaggatccaggtctcccgacgggaggttaataccaaga

SEQ ID NO: 2 - DNA, 1063 bp, human
tgcaaagcctttctacatccttccactatatggaacccccaaaccacaactgtggcacttttattttaattatttttattatttatttatttatgtatttatctcttgaggtggcctcgctctgtcactcaggctggagtgcagtggagcaatcacggttcaaggcgcctcgatctccgatccccggggctcaagcgatcctccctcctcagcctccggagctggagttacaggtgtgcgatgcctcgcctggctattttttttcctttttgggtagagacggggtctcgctatgttgcccaggctggtctccaactcctaggctccagcgatcctcccgcctcggcctcccaatgtgctgcgaatacagactccagccaccgcacacagcctacttttatttctttgaaaaatgaattcgagggtaaagggggcggggttgaggcagatgccagaatctgttcgcttcaaccaagcagccaggctgcctgtccagaaagccggcactcagtttcctcaggaaaacgaagctaaggctcccattcccctcgctaacaacgtcagaacagaggacagtttttagatttcagggatcttaaatagattggcagttcctggagaataaacatcctttgcttttctcctgcacacttttgcctcaggccaccccttcccgcttccaaagcccatctcttccaagctttccgcacgagaaaacaagtgagcccttctcattggccagattccctgtcaatctctccgctatgacgccgagtggtgccttttgaagcctctctagtcccgcctccctaacctgattggtttattcaaacaaaccccggccaactcagccgttcataggtggatataaaaggcaagctacgattggttcttctggacggagacggtgagagcgagtcagggattggctggtctgcttcgggcgggctaaaggaaggttcaagtggagctctcctaaccgacgcgcgtctgtggagaagcggcttggtcgggggtggtctcgtggggtcctgcctgtttagtcgctttcagggttcttgag

SEQ ID NO: 3 - protein, 20 aa, artificial
  Nucleic acid condensing peptid
RQRQRYYRQRQRGGRRRRRR

SEQ ID NO: 4 - protein, 13 aa, artificial
  Nucleic acid condensing peptid
RQRQRGGRRRRRR

SEQ ID NO: 5 - protein, 21 aa, artificial
  Nucleic acid condensing peptid
RRRRRRYYRQRQRGGRRRRRR

SEQ ID NO: 6 - protein, 39 aa, artificial
  Nucleic acid condensing peptid
GNQSSNFGPMKGGNFGGRSSGPYGGGGQYFAKPRNQGGY

SEQ ID NO: 7 - DNA, 44 bp, artificial
  Forward Primer
tatcgataggtaccgagctctaaagttgcctttcaagctctggc

SEQ ID NO: 8 - DNA, 40 bp, artificial
  Reverse Primer
gatcgcagatctcgagtcttggtattaacctcccgtcggg

SEQ ID NO: 9 - DNA, 44 bp, artificial
  Forward Primer
tatcgataggtaccgagctctgcaaagcctttctacatccttcc

SEQ ID NO: 10 - DNA, 40 bp, artificial
  Reverse Primer
gatcgcagatctcgagctcaagaaccctgaaagcgactaa

(19) United States
(12) Patent Application Publication
(10) Pub. No.: US 2021/0404021 A1
(43) Pub. Date: Dec. 30, 2021

(54) METHOD, KIT, AND PROGRAM FOR DETERMINING CHARACTERISTIC OF TUMOR CELL GROUP

(71) Applicant: KABUSHIKI KAISHA TOSHIBA, Tokyo (JP)

(72) Inventors: Shigehisa KAWATA, Niiza (JP); Mitsuko ISHIHARA, Setagaya (JP); Saeko SARUWATARI, Kawasaki (JP)

(73) Assignee: KABUSHIKI KAISHA TOSHIBA, Tokyo (JP)

(21) Appl. No.: 17/470,722

(22) Filed: Sep. 9, 2021

Related U.S. Application Data
(63) Continuation of application No. PCT/JP2019/042047, filed on Oct. 25, 2019.

(51) Int. Cl. C12Q 1/6897; C12Q 1/6886

(52) U.S. Cl. CPC C12Q 1/6897; C12Q 1/6886

(57) ABSTRACT

According to one embodiment, a method for determining a characteristic of a tumor cell group, the method includes bringing a lipid particle including a lipid membrane and a first nucleic acid included in the lipid membrane into contact with the tumor cell group (the first nucleic acid including a promoter sequence of a marker gene for determining the characteristic and a reporter gene linked to a downstream of the promoter sequence to be functionable), culturing the tumor cell group, detecting the presence or absence or an amount of a signal from a reporter protein that is expressed from the reporter gene, in each tumor cell included in the tumor cell group, and counting the number of tumor cells having the characteristic that is measured from a result of the detecting.

### CROSS-REFERENCE TO RELATED APPLICATIONS

This application is a Continuation Application of PCT Application No. PCT/JP2019/042047, filed Oct. 25, 2019, the entire contents of which are incorporated herein by reference.

### FIELD

Embodiments described herein relate generally to a method, a kit, and a program for determining a characteristic of a tumor cell group.

### BACKGROUND

The number of breast cancer patients has increased every year. In order to reduce the cost of medical care and to reduce the burden of a patient and a medical service worker, it has been required to accurately predict drug sensitivity of a breast cancer. Recently, various markers for predicting the drug sensitivity of the breast cancer have been found.

For example, the drug sensitivity of the breast cancer can be predicted by detecting the amount of transcripts (mRNA) of a marker gene in a breast cancer cell that is sampled from a patient with a PCR method or a microarray method.

### BRIEF DESCRIPTION OF THE DRAWINGS

FIG. 1 is a flowchart illustrating an example of a method of a first embodiment.

FIG. 2 is a sectional view illustrating an example of a lipid particle of the first embodiment.

FIG. 3 is a schematic view illustrating an example of a first nucleic acid of the first embodiment.

FIG. 4 is a flowchart illustrating an example of the method of the first embodiment.

FIG. 5 is a schematic view and a graph illustrating a manner in which a signal is obtained in the method of the first embodiment.

FIG. 6 is a flowchart illustrating an example of a method of a second embodiment.

FIG. 7 is a flowchart illustrating an example of the method of the second embodiment.

FIG. 8 is a schematic view illustrating an example of a first nucleic acid that is used in a third embodiment.

FIG. 9 is a schematic view illustrating an example of the first nucleic acid that is used in the third embodiment.

FIG. 10 is a flowchart illustrating an example of a method of the third embodiment.

FIG. 11 is a flowchart illustrating an example of the method of the third embodiment.

FIG. 12 is a flowchart illustrating an example of a method of a fourth embodiment.

FIG. 13 is a flowchart illustrating an example of the method of the fourth embodiment.

FIG. 14 is a perspective view illustrating an example of a state when a cell culture and detection device of a fifth embodiment is used.

FIG. 15 is an enlarged view of a sectional surface obtained by cutting an optical sensor illustrated in FIG. 14 along A-A′.

FIG. 16 is a block diagram illustrating an example of a cell characteristic determination system of the fifth embodiment.

FIG. 17 is a schematic view illustrating a vector used in Example 1.

FIG. 18 is a schematic view illustrating a vector used in Example 1.

FIG. 19 is a graph illustrating an experimental result of Example 3.

FIG. 20 is a graph illustrating an experimental result of Example 3.

FIG. 21 is a microscope photograph (a JPEG format) illustrating an experimental result of Example 4.

FIG. 22 is a microscope photograph (a bitmap format) illustrating an experimental result of Example 4.

### DETAILED DESCRIPTION

In general, according to one embodiment, a method is determining a characteristic of a tumor cell group. The method comprises bringing a lipid particle including a lipid membrane and a first nucleic acid included in the lipid membrane into contact with the tumor cell group, wherein the first nucleic acid includes a promoter sequence of a marker gene for determining the characteristic and a reporter gene linked to a downstream of the promoter sequence to be functionable, culturing the tumor cell group, detecting the presence or absence or an amount of a signal from a reporter protein that is expressed from the reporter gene, in each tumor cell included in the tumor cell group, and counting the number of tumor cells having the characteristic that is measured from a result of the detecting.

Hereinafter, embodiments will be described with reference to the accompanying drawings. Note that, in each embodiment, substantially the same constituent parts are denoted by the same reference signs and an explanation thereof will be partly omitted in some cases. The drawings are schematic, and a relation of thickness and planer dimension of each part, a thickness ratio among parts, and so on are sometimes different from actual ones.

A method according to one embodiment is the method to predict a characteristic of a tumor cell. The method comprises bringing a lipid particle including a lipid membrane and a first nucleic acid included in the lipid membrane into contact with the tumor cell group, wherein the first nucleic acid including a promoter sequence of a marker gene for determining the characteristic and a reporter gene linked to a downstream of the promoter sequence to be functionable, culturing the tumor cell group, detecting the presence or absence or an amount of a signal from a reporter protein that is expressed from the reporter gene, in each tumor cell included in the tumor cell group, and counting the number of tumor cells having the characteristic that is measured from a result of the detecting.

Hereinafter, each embodiment will be described.

### First Embodiment

(Method for Determining Sensitivity of Breast Cancer Cell Group with Respect to Drug)

A method according to a first embodiment is a method for determining the sensitivity of a breast cancer cell group with respect to a drug.

A breast cancer according to the embodiment, for example, indicates a malignant tumor (a neoplasm) formed on a mammary gland of an animal. The breast cancer is generally referred to as a “cancer of the breast”, a “mammary gland tumor”, or the like, and includes a ductal breast cancer and a lobular cancer. In addition, the breast cancer also includes a breast cancer in any disease stage, and for example, includes a state in which the cancer remains on the mammary gland, a state in which the cancer further spreads to the surrounding tissue, a state in which the cancer metastasis further occurs in a lymph node, a state in which the cancer metastasis further occurs in a separate internal organ, and the like.

The breast cancer cell group is preferably a breast cancer cell group sampled from a target diseased with a breast cancer, and includes a plurality of breast cancer cells. The breast cancer cell group, for example, is included in a specimen that is obtained by aspirating an affected area of the breast cancer with a needle (fine-needle aspiration, a needle biopsy, a mammotome biopsy, or the like), by surgical excision, or by sampling mammary gland secretion (breast fluid).

The target, for example, is a mammal, and is preferably a human.

The breast cancer cell group may be subjected to a suitable treatment after being sampled. The suitable treatment, for example, is shredding, dispersion, the removal of other cell groups, the isolation of the breast cancer cell group, and the like. Alternatively, the breast cancer cell group may be culture cells obtained by culturing the sampled or pretreated breast cancer cell group in a manner where the characteristic of the breast cancer is not lost. Alternatively, the breast cancer cell group may be an established breast cancer cell group or the like. Hereinafter, the “breast cancer cell group” will be also simply referred to as a “cell group”. In addition, the “breast cancer cell” will be also simply referred to as a “cell”.

The sensitivity of the cell group with respect to the drug indicates whether or not the cell group receives a treatment effect of the drug or a degree of receiving the treatment effect. For example, the cell group having sensitivity with respect to a specific drug indicates that the cell group receives a treatment effect of the drug, and for example, indicates that the cells included in the cell group are decreased or extinguished by the drug, or the proliferation of the cells is suppressed. On the contrary, the cell group not having sensitivity with respect to a specific drug indicates that the cell group has resistiveness (resistance properties) with respect to the drug, and there is no or little treatment effect.

The drug, for example, can be selected from drugs having antitumor activity that are generally used in a medical treatment of a breast cancer. For example, the drug is selected from any of an anticancer agent, a hormone therapeutic agent, a molecularly targeted drug, an immunomodulator, and the like. The anticancer agent, for example, includes an anthracycline-based drug, an antimicrotubule drug, an alkylating compound, an antimetabolic drug, a platinum complex, and the like, and the hormone therapeutic agent, for example, includes an aromatase inhibitor, an LH-RH agonist formulation, an anti-estrogen drug, a progestational agent, and the like. However, the drug is not limited to the above.

For example, in the method according to the embodiment, one type of drug for which the sensitivity in the breast cancer cell group is examined is selected from any of the above. Alternatively, as described in a third embodiment, a plurality of types of drugs may be selected. Hereinafter, an example will be described in which a drug is examined.

A method for determining the sensitivity of the breast cancer cell according to the embodiment with respect to the drug, for example, includes the following steps illustrated in FIG. 1.

(S1) a contact step of bringing a lipid particle including a lipid membrane and a first nucleic acid included in the lipid membrane into contact with a breast cancer cell group (the first nucleic acid includes a promoter sequence of a marker gene for determining sensitivity of a breast cancer cell with respect to a drug and a reporter gene linked to a downstream of the promoter sequence to be functionable);

(S2) a culture step of culturing the breast cancer cell group;

(S3) a detection step of detecting the presence or absence or the amount of a signal from a reporter protein that is expressed from the reporter gene, in each breast cancer cell included in the breast cancer cell group; and

(S4) a counting step of measuring the number of tumor cells having drug sensitivity from a result of the detection step.

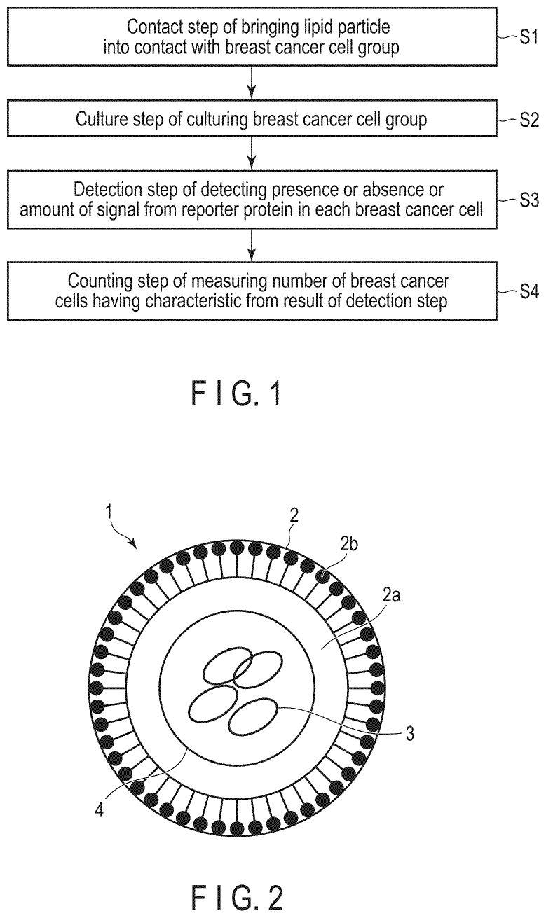

First, the lipid particle that is used in the contact step S1 will be described. As illustrated in FIG. 2, a lipid particle 1, for example, includes a lipid membrane 2 and a first nucleic acid 3 included in an inner cavity 2a of the lipid membrane 2. The first nucleic acid 3, for example, is included in the lipid membrane 2 that is condensed by nucleic acid condensed peptide 4. The details will be described below, and the nucleic acid condensed peptide 4 is used as a carrier for introducing the first nucleic acid 3 into the breast cancer cell.

The lipid membrane 2 is approximately a spherical hollow body that is formed by sequencing a plurality of lipid molecules 2b with a non-covalent bond. The lipid membrane 2 may be a lipid monomolecular membrane, or may be a lipid bilayer membrane. In addition, the lipid membrane 2 may include a one-layer membrane, or may include a multi-layer membrane.

A lipid that is the material of the lipid membrane 2, for example, includes a lipid that is a main component of a biological membrane (hereinafter, referred to as a “base lipid”). The base lipid is a phospholipid or a sphingolipid, and for example, is diacyl phosphatidyl choline, diacyl phosphatidyl ethanol amine, ceramide, sphingomyelin, dihydrosphingomyelin, kephalin, cerebroside, a combination thereof, and the like.

For example, it is preferable to use
1,2-dioleoyl-sn-glycero-3-phosphoethanol amine (DOPE),
1,2-stearoyl-sn-glycero-3-phosphoethanol amine (DSPE),
1,2-dipalmitoyl-sn-glycero-3-phosphatidyl choline (DPPC),
1-palmitoyl-2-oleoyl-sn-glycero-3-phosphatidyl choline (POPC),
1,2-di-O-octadecyl-3-trimethyl ammonium propane (DOTMA),
1,2-dioleoyl-3-dimethyl ammonium propane (DODAP),
1,2-dimyristoyl-3-dimethyl ammonium propane (14:0 DAP),
1,2-dipalmitoyl-3-dimethyl ammonium propane (16:0 DAP),
1,2-distearoyl-3-dimethyl ammonium propane (18:0 DAP),
N-(4-carboxybenzyl)-N,N-dimethyl-2,3-bis(oleoyloxy)propane (DOBAQ),
1,2-dioleoyl-3-trimethyl ammonium propane (DOTAP),
1,2-dioleoyl-sn-glycero-3-phosphochlorine (DOPC),
1,2-dilinoleoyl-sn-glycero-3-phosphochlorine (DLPC),
1,2-dioleoyl-sn-glycero-3-phospho-L-serine (DOPS), cholesterol,

any combination thereof, and the like, as the base lipid.

It is preferable that the lipid membrane 2 further contains any one of both of a first lipid compound and a second lipid compound described below, in addition to the base lipid.

The first lipid compound can be represented by the formula of Q-CHR₂.

(In the formula,

Q is a nitrogen-containing aliphatic group that contains two or more tertiary nitrogens but does not contain oxygen,

Rs are each independently an aliphatic group having C₁₂ to C₂₄, and

at least an R includes a linking group LR selected from the group consisting of —C(═O)—O—, —O—C(═O)—, —O—C(═O)—O—, —S—C(═O)—, —C(═O)—S—, —C(═O)—NH—, and —NHC(═O)—, in a main chain or a side chain thereof).

For example, it is preferable to use lipids having structures represented by the following formulas as the first lipid compound since an introduction efficiency of a nucleic acid is more excellent.

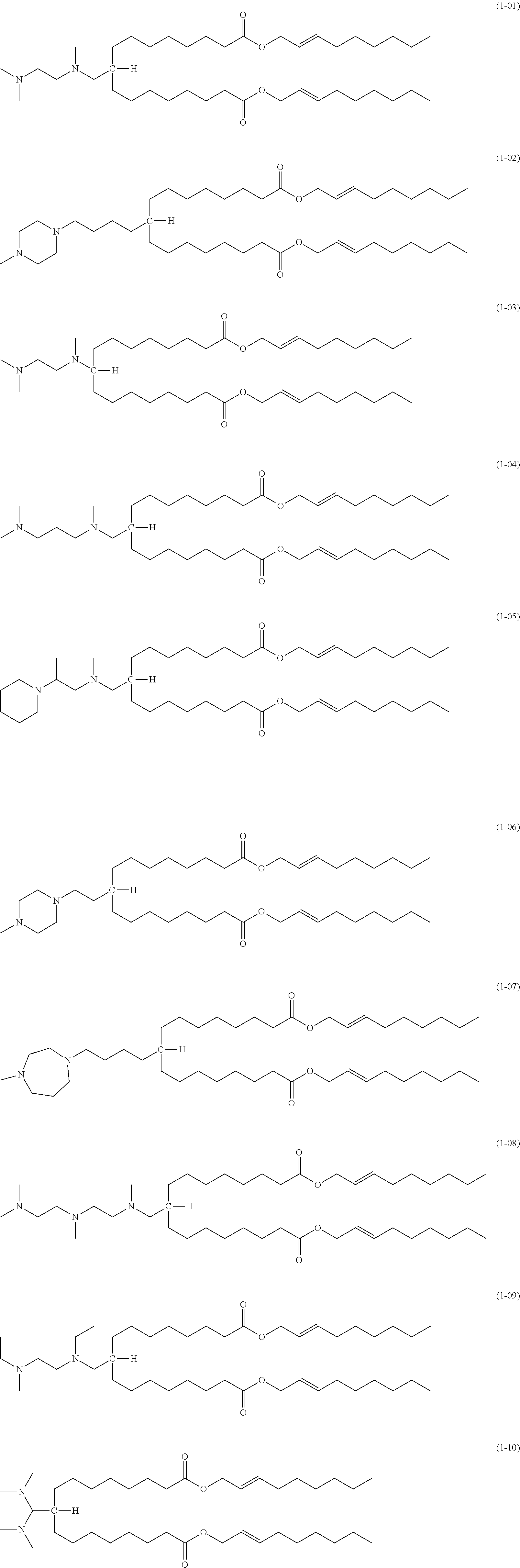

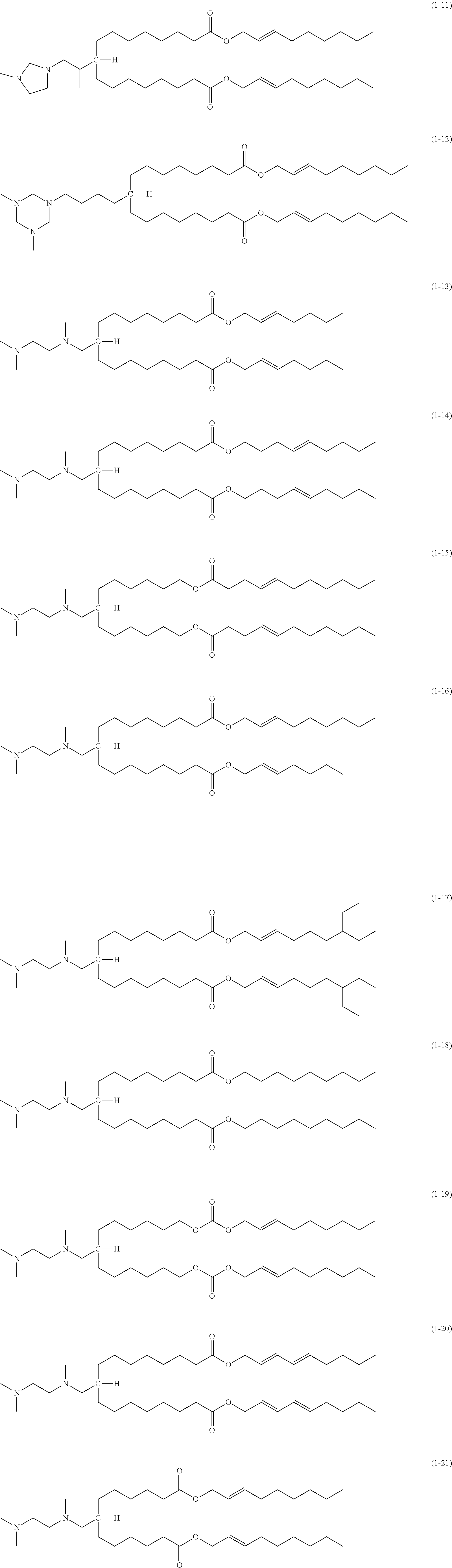

The second lipid compound can be represented by the formula of P—[X—W—Y—W′—Z]₂.

(In the formula,

P is alkylene oxide having one or more ether bonds in a main chain,

Xs are each independently a divalent linking group having a tertiary amine structure,

Ws are each independently alkylene having C₁ to C₆,

Ys are each independently a divalent linking group selected from the group consisting of a single bond, an ether bond, a carboxylic ester bond, a thiocarboxylic ester bond, a thioester bond, an amide bond, a carbamate bond, and a urea bond,

W's are each independently a single bond or alkylene having C₁ to C₆, and

Zs are each independently a fat-soluble vitamin residue, a sterol residue, or an aliphatic hydrocarbon group having C₁₂ to C₂₂).

In the case of containing the second lipid compound, oxygen configuring the ether bond included in P forms a hydrogen bond with the nucleic acid to be included, and thus, an inclusion amount of the nucleic acid increases.

For example, it is preferable to use second lipid compounds having the following structures since the inclusion amount of the nucleic acid is more excellent.

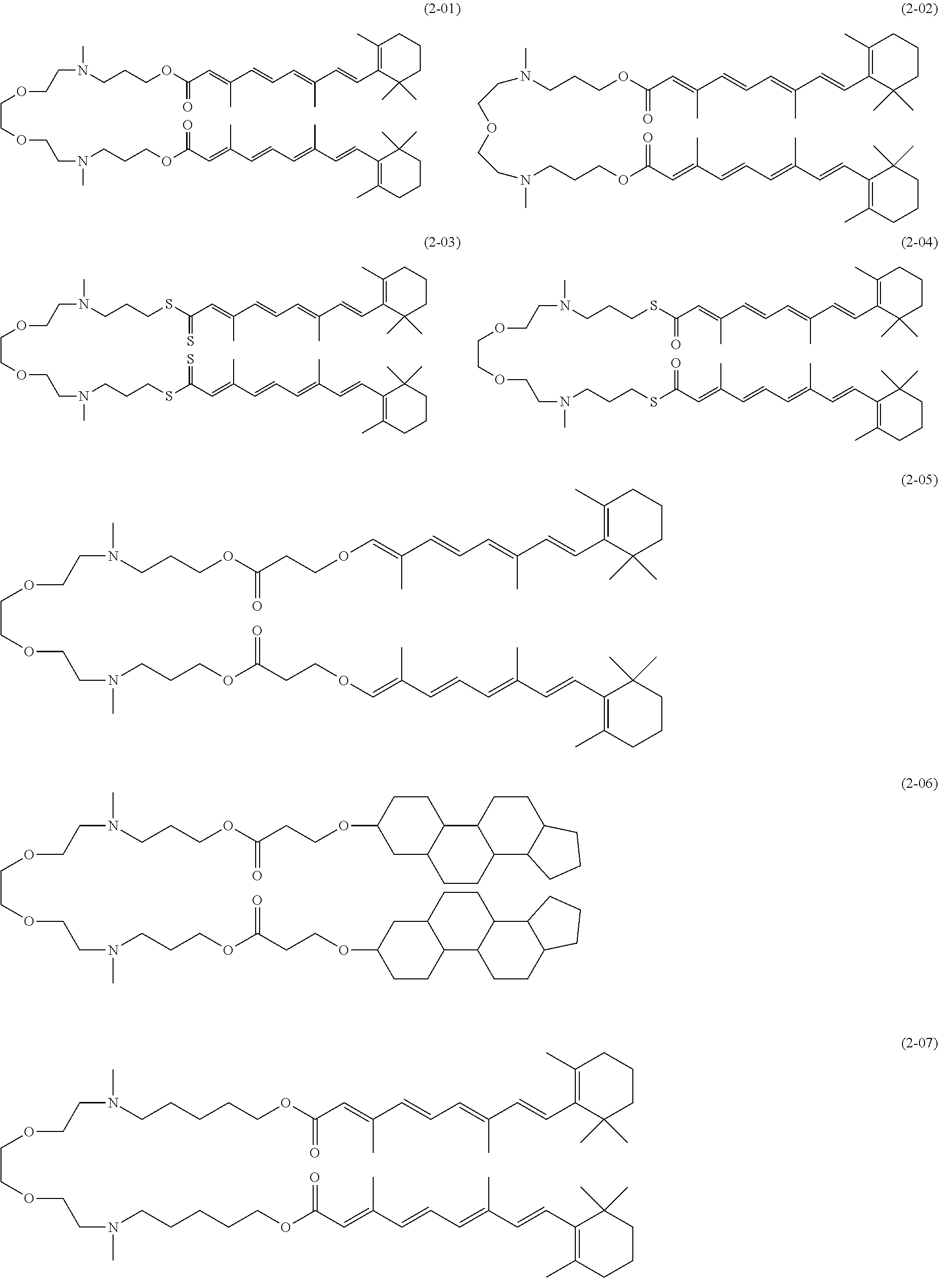

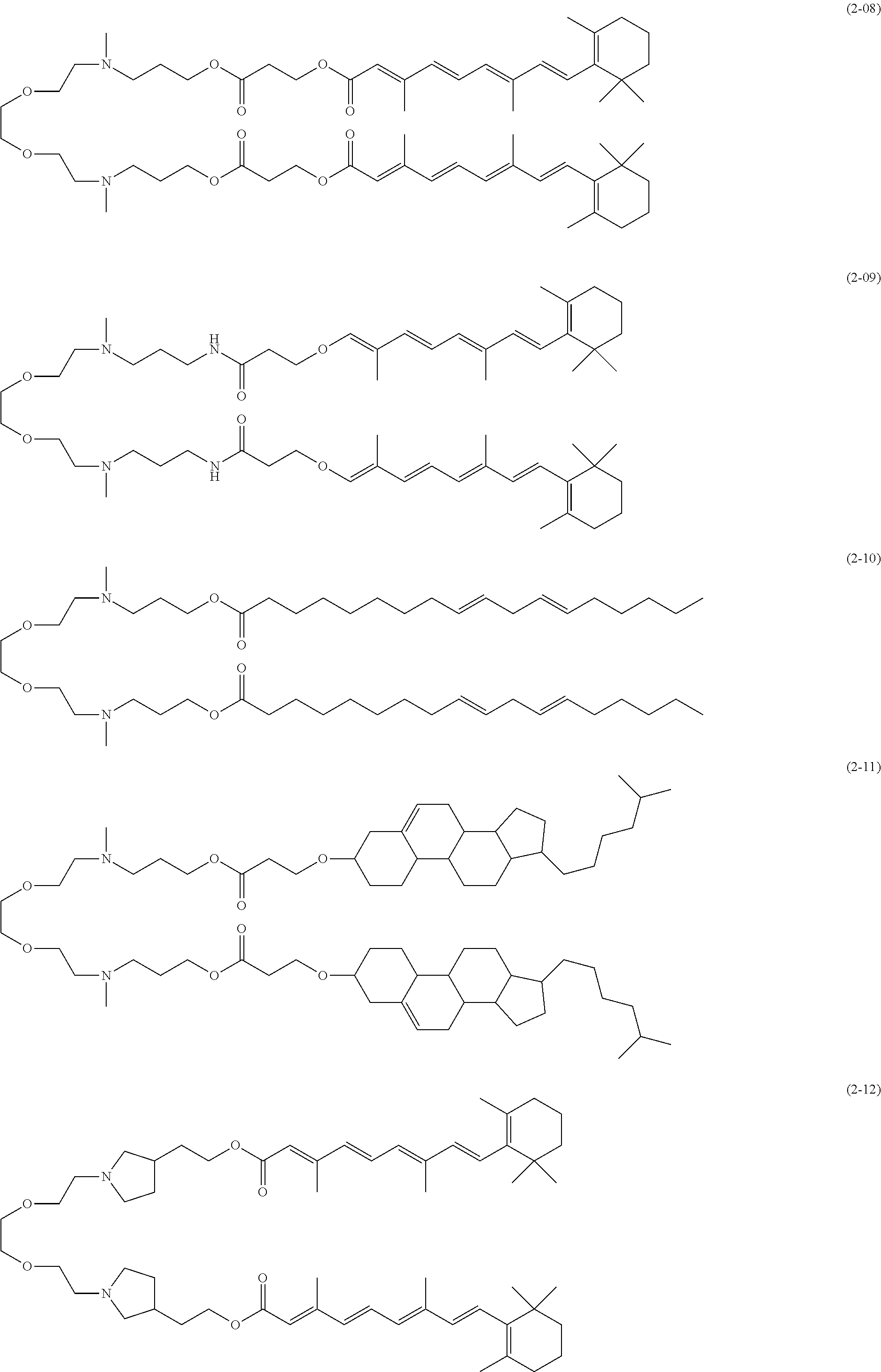

Approximately 100% of all of the lipid molecules 2b contained in the lipid membrane 2 may be the base lipid, and it is preferable that the first lipid compound and/or the second lipid compound are contained at approximately 20% to approximately 70% (a molar ratio) with respect to all of the lipid molecules 2b.

It is preferable that the lipid membrane 2, for example, further contains a PEG-modified lipid, in particular, polyethylene glycol (PEG) dimyristoyl glycerol (DMG-PEG), a polyamide oligomer derived from an omega-amino(oligoethylene glycol) alkanoic acid monomer (for example, described in U.S. Pat. No. 6,320,017 B), and monosialoganglioside. It is preferable that such a lipid is contained at approximately 1% to approximately 5% (a molar ratio) with respect to all of the lipid molecules 2b.

The lipid membrane 2 may contain a lipid having relatively high biological compatibility for adjusting biological compatibility of the lipid particle 1, a lipid having a functional group for bonding a ligand to the lipid membrane 2, a lipid for suppressing the leakage of an inclusion such as cholesterol, and the like.

In particular, it is more preferable to use the first lipid compound having Formula (1-01) described above and/or the second lipid compound having Formula (2-01) described above since the first nucleic acid 3 is easily introduced to the breast cancer cell. For example, it is preferable that the lipid particle 1 contains a compound of Formula (1-01), a compound of Formula (2-01), DOTAP, DOPE, cholesterol, and DMG-PEG since the introduction efficiency with respect to the breast cancer cell is particularly excellent. For example, it is more preferable that such components are contained at 37:15:10.5:10.5:30:2 (a molar ratio).

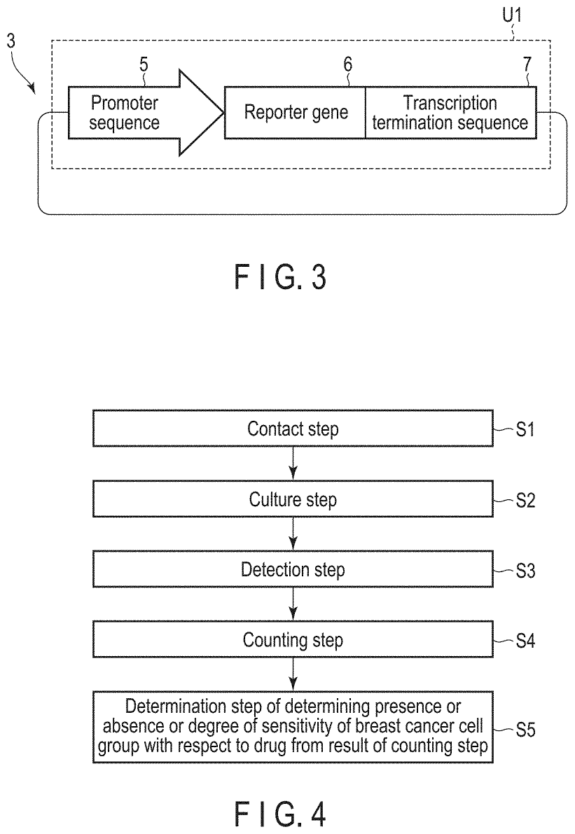

For example, as illustrated in FIG. 3, the first nucleic acid 3 includes a first reporter expression unit U1. The first reporter expression unit U1, for example, includes a promoter sequence 5, a reporter gene 6, and a transcription termination sequence 7.

The promoter sequence 5 is a promoter sequence of a marker gene for determining the sensitivity of the breast cancer cell with respect to the drug.

The marker gene is selected corresponding to the type of drug that is examined in this method. The marker gene, for example, is a gene of which an expression amount is different between a breast cancer cell having the sensitivity with respect to the drug and a breast cancer cell not having the sensitivity with respect to the drug. Alternatively, the marker gene may be a gene of which an expression amount is changed in accordance with the existence of the selected drug, in the breast cancer cell. A change in the expression amount includes both of an increase in the expression amount and a decrease in the expression amount.

For example, a gene that is known in this field as a drug sensitivity predictive marker, a treatment effect predictive marker, a drug selective marker, or the like can also be used as the marker gene.

For example, in a case where the drug is the anthracycline-based drug, TOP2A, RARA, CDCl₆, THRA, GSDM1, PSMD3, CSF3, MED24, SNORD124, NR1D1, TRNASTOP-UCA, MSL-1, CASC3, RAPGEFL1, WIPF2, LOC100131821, GJD3, LOC390791, LOC728207, IGFBP4, TNS4, CCR7, SMARCE1, or the like can be used as the marker gene.

For example, in a case where the drug is the aromatase inhibitor, STC2, SLC39A6, CA12, ESR1, PDZK1, NPY1R, CD2, MAPT, QDPR, AZGP1, ABAT, ADCY1, CD3D, NAT1, MRPS30, DNAJC12, SCUBE2, KCNE4, DHA, ATP5J2, VDAC2, DARS, UCP2, UBE2Z, AK2, WIPF2, APPBP2, TRIM2, or the like can be used as the marker gene.

However, the marker gene is not limited to the above.

Alternatively, the gene of which the expression amount is different between the breast cancer cell having the sensitivity with respect to the drug and the breast cancer cell not having the sensitivity with respect to the drug or the gene of which the expression amount is changed when the drug is added to the breast cancer cell may be examined and used as the marker gene.

The entire length of the promoter sequence of the marker gene may be used as the promoter sequence 5, and a part thereof or an altered sequence may be used unless the promoter activity thereof is affected. It is preferable that the promoter sequence 5 is shorter from the viewpoint of stability in the lipid particle 1 or the inclusion amount.

For example, in a case where the stanniocalcin 2 (STC2) is used as the marker gene, it is preferable that a sequence of Table 1 described below (SEQ ID NO: 1) is used as the promoter sequence.

TABLE 1
STC2 promoter sequence
(SEQ ID NO: 1)

TAAAGTTGCCTTTCAAGCTCTGGCCTCCGGGCACGC
GATGCTCCGCGGCGGGCTGACTCAGGGCTGCCTTG
GGCCTCCCTGCCACCCTCCTGGAAATGATGCAAGT
CCTGACTGTCACCTGGATCCCTGCAGCCCAGCCTG
GAATGCGTCTGGATTAGGGGAAAGACGAGAAACGA
CACTCCAGGTGTTGCACGGCCCACCAAAGCGGGAA
GATAGGGCAGTTGCTCAGACCAAATACTGTATCTA
GTGCTTCTGCTCCTATCTTCAATCGTGGGGTTCTT
TTTAATGCAAAGTGTCACAAGGCCAGGAATTCCCA
TGTGTGCTCAGTTGGCCCACAGCATCATTGTGCCT
AGGAAACTGCTTCAATTTATCAAGTCCTCTGGGCT
GGGAATCTCACTGAATTCCAAACGGCGGAAAGAGG
AAACTTTCCCAACCCGATGTGGGTGTGACGCGAGC
CAGGGGCCCCAGGGACACTGTCCCAGAGCACACCG
TCCCCCTTTAACAGCAACTGGAGCTTGGATTCGCT
CTTATATTGTACAGTCCTTTCGACCATTGCCCTGG
AGCACCCGCACACGCGCACGCATCTCCGGCCGCGC
TCACACACACTCATACACACGCACGCAAACGCGTG
GCCGCCGCCAGGTCGGCAACTTTGTCCGGCGCTCC
CAGCGGCGCTCGGCTTCCTCCTGTAGTAGTTGAGC
GCAGGCCCCGCCTCCCGGCCGTGTTGTCAAAAGGG
CCGGGGTCTCGGATTGGTCCAGCCGCCGGGACAAC
ACCTGCTCGACTCCTTCATTCAAGTGACACCAGAG
CTTCCAGGGATATTTGAGGCACCATCCCTGCCATT
GCCGGGCACTCGCGGCGCTGCTAACGGCCTGGTCA
CATGCTCTCCGGAGAGCTACGGGAGGGCGCTGGGT
AACCTCTATCCGAGCCGCGGCCGCGAGGAGGAGGG
AAAAGGCGAGCAAAAAGGAAGAGTGGGAGGAGGAG
GGGAAGCGGCGAAGGAGGAAGAGGAGGAGGAGGAA
GAGGGGAGCACAAAGGATCCAGGTCTCCCGACGGG
AGGTTAATACCAAGA

For example, in a case where the topoisomerase 2 alpha (TOP2A) is used as the marker gene, it is preferable that a sequence of Table 2 described below (SEQ ID NO: 2) is used as the promoter sequence.

TABLE 2
TOP2A promoter sequence
(SEQ ID NO: 2)

TGCAAAGCCTTTCTACATCCTTCCACTATATGGAAC
CCCCAAACCACAACTGTGGCACTTTTATTTTAATT
ATTTTTATTATTTATTTATTTATGTATTTATCTCT
TGAGGTGGCCTCGCTCTGTCACTCAGGCTGGAGTG
CAGTGGAGCAATCACGGTTCAAGGCGCCTCGATCT
CCGATCCCCGGGGCTCAAGCGATCCTCCCTCCTCA
GCCTCCGGAGCTGGAGTTACAGGTGTGCGATGCCT
CGCCTGGCTATTTTTTTTCCTTTTTGGGTAGAGAC
GGGGTCTCGCTATGTTGCCCAGGCTGGTCTCCAAC
TCCTAGGCTCCAGCGATCCTCCCGCCTCGGCCTCC
CAATGTGCTGCGAATACAGACTCCAGCCACCGCAC
ACAGCCTACTTTTATTTCTTTGAAAAATGAATTCG
AGGGTAAAGGGGGCGGGGTTGAGGCAGATGCCAGA
ATCTGTTCGCTTCAACCAAGCAGCCAGGCTGCCTG
TCCAGAAAGCCGGCACTCAGTTTCCTCAGGAAAAC
GAAGCTAAGGCTCCCATTCCCCTCGCTAACAACGT
CAGAACAGAGGACAGTTTTTAGATTTCAGGGATCT
TAAATAGATTGGCAGTTCCTGGAGAATAAACATCC
TTTGCTTTTCTCCTGCACACTTTTGCCTCAGGCCA
CCCCTTCCCGCTTCCAAAGCCCATCTCTTCCAAGC
TTTCCGCACGAGAAAACAAGTGAGCCCTTCTCATT
GGCCAGATTCCCTGTCAATCTCTCCGCTATGACGC
CGAGTGGTGCCTTTTGAAGCCTCTCTAGTCCCGCC
TCCCTAACCTGATTGGTTTATTCAAACAAACCCCG
GCCAACTCAGCCGTTCATAGGTGGATATAAAAGGC
AAGCTACGATTGGTTCTTCTGGACGGAGACGGTGA
GAGCGAGTCAGGGATTGGCTGGTCTGCTTCGGGCG
GGCTAAAGGAAGGTTCAAGTGGAGCTCTCCTAACC
GACGCGCGTCTGTGGAGAAGCGGCTTGGTCGGGGG
TGGTCTCGTGGGGTCCTGCCTGTTTAGTCGCTTTC
AGGGTTCTTGAG

The reporter gene 6 is linked to the downstream of the promoter sequence 5 to be functionable. Being linked to be functionable indicates that the reporter gene 6 expresses a reporter protein by the promoter activity of the promoter sequence 5.

A gene that is generally used in a reporter assay can be used as the reporter gene 6. The reporter gene 6 is a gene coding the reporter protein that generates a detectable signal, and for example, is preferably a gene coding a luminescent enzyme protein, such as a firefly luciferase gene, a sea pansy luciferase gene, or a NanoLuc (Registered Trademark) luciferase gene, a gene coding a fluorescent protein, such as a green fluorescent protein gene, a blue fluorescent protein gene, or a red fluorescent protein gene, or the like. Alternatively, the reporter gene 6 may be a drug-resistant gene such as an ampicillin-resistant gene or a kanamycin-resistant gene; a gene coding an activity oxygen producing enzyme, such as a xanthine oxidase gene or a nitric oxide synthetase gene; a gene coding a color-producing enzyme protein, such as a β-galactosidase gene or a chloramphenicol acetyltransferase gene; a gene coding a heavy metal-binding protein, or the like.

The transcription termination sequence 7, for example, may be a sequence including a polyadenylation signal sequence that terminates the transcription of the reporter gene 6, and for example, can be a polyadenylation signal sequence. For example, a polyadenylation signal sequence of a simian virus (SV) 40, a polyadenylation signal sequence of a bovine growth hormone gene, a polyadenylation signal sequence that is altered or artificially synthesized insofar as a function of terminating the transcription of such a sequence is maintained, and the like can be used as the polyadenylation signal sequence.

The first nucleic acid 3, for example, is a circular double-stranded DNA. The first nucleic acid 3 may be a vector. In a case where the first nucleic acid 3 is circular, it is possible for the first nucleic acid 3 to stably exist in the lipid particle 1, and the decomposition or the damage of the first nucleic acid 3 is prevented. Alternatively, the first nucleic acid 3 may be a linear nucleic acid. In a case where the first nucleic acid 3 is linear, the size of the first nucleic acid 3 can be reduced, compared to a case in which the first nucleic acid 3 is circular, and thus, more first nucleic acids 3 can be included in the lipid membrane 2.

The first nucleic acid 3 may include an additional sequence, in addition to the sequences described above. For example, in a case where the first nucleic acid 3 is the vector, the first nucleic acid 3 may include a replication initiator sequence for replicating the first nucleic acid 3 itself, a sequence for expressing a gene coding a replication initiator protein involved in replication initiation, and the like.

The nucleic acid condensed peptide 4, for example, is a cationic peptide. A preferred nucleic acid condensed peptide 4, for example, is a peptide containing a cationic amino acid at greater than or equal to 45% of the total. A more preferred nucleic acid condensed peptide 4 includes RRRRRR (a first amino acid sequence) on one end, and includes a sequence RQRQR (a second amino acid sequence) on the other end. Then, the nucleic acid condensed peptide 4 includes 0 or one or more intermediate sequences including RRRRRR or RQRQR between both of the amino acid sequences. In addition, the nucleic acid condensed peptide 4 contains two or more neutral amino acids between two adjacent sequences, in the first amino acid sequence, the second amino acid sequence, and the intermediate sequence. The neutral amino acid, for example, is G or Y.

The nucleic acid condensed peptide 4 described above preferably includes the following amino acid sequences.

(SEQ ID NO: 3)
RQRQRYYRQRQRGGRRRRRR
(SEQ ID NO: 4)
RQRQRGGRRRRRR.

Alternatively, the nucleic acid condensed peptide 4 includes RRRRRR (a third amino acid sequence) on one end, and includes RRRRRR (a fourth amino acid sequence) on the other end. Then, the nucleic acid condensed peptide 4 includes 0 or one or more intermediate sequences including RRRRRR or RQRQR between both of the amino acid sequences. In addition, the nucleic acid condensed peptide 4 contains two or more neutral amino acids between two adjacent sequences, in the third amino acid sequence, the fourth amino acid sequence, and the intermediate sequence.

Such a nucleic acid condensed peptide 4 preferably includes the following amino acid sequence.

(SEQ ID NO: 5)
RRRRRRYYRQRQRGGRRRRRR.

Further, a nucleic acid condensed peptide 4 including the following amino acid sequence can also be used by being combined with any of the nucleic acid condensed peptides 4 described above.

(SEQ ID NO: 6)
GNQSSNFGPMKGGNFGGRSSGPYGGGGQYFAKPRNQGGY
(M9)

In the case of using the nucleic acid condensed peptide 4, the first nucleic acid 3 is condensed, more first nucleic acids 3 can be included in the lipid membrane 2, and the size of the lipid particle 1 can be reduced. As a result thereof, the introduction efficiency of the first nucleic acid 3 with respect to the breast cancer cell increases. It is preferable to use the nucleic acid condensed peptide 4, but the nucleic acid condensed peptide 4 may not be used in accordance with the type of nucleic acid to be used, or the like.

The lipid particle 1 may include additional components, in addition to the first nucleic acid 3. For example, the lipid particle 1 is also capable of including a compound adjusting the expression of a nucleic acid in a cell, such as a retinoic acid, a circular adenosine monophosphate (cAMP), or an ascorbic acid; peptide, polypeptide, cytokine, a proliferative factor, an apoptotic factor, a differentiation responsive factor, other cell surface receptors, ligands thereof, and the like.

The lipid particle 1 can be manufactured as follows. For example, first, the first nucleic acid 3 and the nucleic acid condensed peptide 4 are stirred and mixed, and thus, the first nucleic acid 3 is condensed. Then, an aqueous buffer containing components to be included in the lipid membrane, such as the first nucleic acid 3, is added to a solution containing a lipid that is the material of the lipid membrane 2, and is stirred and suspended. Alternatively, such preparation can be performed by using a known method that is used at the time of encapsulating the lipid particle or the like with small molecules, for example, a Bangham method, an organic solvent extraction method, a surfactant removal method, a freezing and thawing method, or the like. The inclusion amount of the first nucleic acid 3 can be checked, for example, by using commercially available DNA and RNA quantitative kits, or the like.

An average particle diameter of the lipid particle 1, for example, is approximately 50 nm to approximately 300 nm, and is preferably approximately 50 nm to approximately 200 nm. For example, a particle diameter can be decreased by an ultrasonic treatment. In addition, the size of the lipid particle 1 can be adjusted by permeating the lipid particle 1 through a polycarbonate membrane, a ceramic membrane, or the like. The average particle diameter of the lipid particle 1, for example, can be measured by Zetasizer using a dynamic light scattering method.

The lipid particle 1, for example, may be prepared as a composition that is contained in a suitable solvent. The solvent, for example, is water, a salt solution such as a normal saline solution, an aqueous glycine solution, a buffer solution, or the like. Alternatively, the lipid particle 1 may be provided in a dried state.

The lipid particle 1 described above is brought into contact with the breast cancer cell group (the contact step S1). For example, the contact step S1 is performed by adding the lipid particle 1 to the breast cancer cell group that is suspended in a suitable solvent or is seeded on a culture medium. Alternatively, the contact step S1 may be performed by a method of adding the breast cancer cell group to a container or the like in which the lipid particle 1 is attached or fixed to the inner surface.

Next, the breast cancer cell group is cultured (the culture step S2). The culture can be performed in a condition advantageous to the existence of the breast cancer cell group, in which the breast cancer cell group maintains properties as a breast cancer. The culture, for example, may be performed on a dish, in a tube, or the like by using a suitable culture medium such as a solid culture medium or a liquid culture medium. It is preferable that the culture is performed at 30 to 37° C. in 2 to 5% CO₂ atmosphere, as a culture condition. It is preferable that a culture time is 2 to 72 hours.

By performing the contact step S1 and the culture step S2 described above, the lipid particle 1 is incorporated into the breast cancer cell included in the breast cancer cell group by endocytosis or the like during the culture step S2, and the first nucleic acid 3 is taken up by the cell. According to the lipid particle 1 of the embodiment, the first nucleic acid 3 can be efficiently and simply introduced to the breast cancer cell.

After that, the promoter sequence 5 of the first nucleic acid 3 is activated in accordance with the activity of a promoter of the marker gene existing in the breast cancer cell, and the reporter gene 6 on the downstream thereof is expressed. Therefore, the activity of the promoter of the marker gene of the breast cancer cell is reflected on the amount of reporter protein, that is, a signal amount generated therefrom, by the first nucleic acid 3. Here, the activity of the promoter of the marker gene indicates an expression frequency of the marker gene, and a strong promoter activity includes high transcription amount and expression amount of the gene, and a high rate of derivation of the expression of the marker gene (an early gene or the like). On the contrary, a weak promoter activity includes low or no transcription amount and expression amount of the gene, and a low rate of derivation of the expression of the marker gene (a late gene or the like).

In a breast cancer cell group, a plurality of types of breast cancer cells in which the presence or absence or the degree of the drug sensitivity is different can be included. For example, in the breast cancer cell group, the signal from the reporter protein is obtained in a breast cancer cell in which a drug sensitivity predictive marker gene is expressed. On the contrary, the signal from the reporter protein is not obtained in a breast cancer cell in which the drug sensitivity predictive marker gene is not expressed. In addition, more signals are obtained in a breast cancer cell in which the activity of the promoter of the marker gene is high.

As described above, it is preferable that the culture time is 2 to 72 hours, and it is preferable to perform the culture until a desired signal amount (the expression amount of the reporter protein) is obtained. More reporter proteins are generated and the signal amount can be increased, as the culture time increases, but after that, the signal amount may be attenuated. For example, the culture time is selected in accordance with the type of marker gene to be used. For example, in the case of a gene having a high promoter activity (the early gene or the like), the culture time may be 2 to 4 hours. On the contrary, in the case of a gene having a low promoter activity (the late gene or the like), it is preferable to perform the culture for 24 to 72 hours.

For example, cells included in the sampled specimen, other than the breast cancer cell, for example, a blood cell, a fibroblast cell, a fat cell, and the like may be killed and removed by the culture. In addition, the breast cancer cell may be proliferated by the culture. In a case where the breast cancer cell that is increased by the culture is also in contact with the lipid particle 1 and the first nucleic acid 3 is introduced, or the breast cancer cell to which the first nucleic acid 3 is introduced is proliferated, the first nucleic acid 3 can also be replicated during division and can be included in each of the divided cells.

In the detection step S3, in each of the breast cancer cells included in the breast cancer cell group, the signal from the reporter protein is individually detected.

In a case where the reporter gene 6 is the gene coding the luminescent enzyme protein, fluorescence from a metabolite obtained by adding a substrate of the luminescent enzyme protein to the breast cancer cell group before the detection and by metabolizing the substrate with the reporter protein (the luminescent enzyme protein) can be a signal. In a case where the reporter gene is the gene coding the fluorescent protein, fluorescence from the reporter protein itself can be a signal.

For example, in order to count the number of cells in which the signal is obtained in the next counting step S4, it is preferable that the signal is detected in each of the breast cancer cells, and the presence or absence or the amount of the signal is individually determined for each of the breast cancer cells.

It is preferable that such detection of the signal is performed by using a detection device that is capable of detecting the presence or absence of the signal for each of the cells, such as an optical sensor, a microscope, a camera, or a cell sorter.

The detection step S3 may be performed after the culture step S2, and it is preferable that the detection step S3 is performed over time while the culture is performed. Accordingly, even in a case where a suitable culture time according to the type of marker gene is unclear, a signal when the signal amount is maximized can be used. As a result thereof, the number of cells in which the signal is accurately obtained can be measured in the subsequent counting step. Being over time may be continuous, or may be intermittent.

Next, the number of breast cancer cells having the drug sensitivity is measured from the result of the detection step S3. For example, in the case of using a marker gene of which an expression amount is positively correlated with the drug sensitivity, a cell in which the signal is obtained (hereinafter, also referred to as a “signal generating cell”) may be regarded to have high promoter activity and may be determined as the cell having the sensitivity with respect to the drug (hereinafter, also referred to as a “drug-sensitive cell”), and the number thereof may be measured. Alternatively, a cell in which the signals of greater than or equal to a threshold value are obtained may be regarded as the signal generating cell, and may be measured as the drug-sensitive cell. The threshold value, for example, can be determined in accordance with the signal amount obtained by introducing the first nucleic acid 3 to the breast cancer cell in which the presence or absence of the drug sensitivity is known.

On the contrary, in the case of using a marker gene of which an expression amount is negatively correlated with the drug sensitivity, a cell in which the signal is not obtained or a cell in which a signal amount that is obtained is less than or equal to the threshold value (hereinafter, also referred to as a “non-signal-generating cell”) can be measured as the drug-sensitive cell.

The counting of the cell may be visually performed from a microscope image or a video that is obtained, or may be performed by using image processing software or the like. Alternatively, the counting of the cell may be automatically performed by using an optical sensor including a plurality of sensor elements described below, a cell sorter, or the like.

Further, the total number of breast cancer cells included in the breast cancer cell group may be measured. For example, an existence rate of the drug-sensitive cell, that is, a ratio of the number of drug-sensitive cells to the total number of breast cancer cells in the breast cancer cell group may be calculated by using the total number of breast cancer cells.

In a case where the reporter gene is the drug-resistant gene, for example, first, the total number of cells included in the breast cancer cell group may be measured, and then, the breast cancer cell group may be treated with a drug corresponding to the drug-resistant gene, the existing breast cancer cell may be detected as the signal, and the number thereof may be measured. The detection and the measurement of the number of cells can be performed by using a detection device such as an optical sensor, a microscope, a camera, or a cell sorter.

In the detection step S3 and the counting step S4, it is not necessary to detect and count the number of signal generating cells in the entire breast cancer cell group. For example, the total number of cells and the number of signal generating cells included in a part of the breast cancer cell group may be counted, and the value thereof may be set to a representative value to be used as the result of the entire breast cancer cell group. For example, the detection step S3 and the counting step S4 may be performed by using an image obtained by capturing a part of the breast cancer cell group, a visual field of a microscope including a part of the breast cancer cell group, or the like. For example, the number of signal generating cells per the number of units of the breast cancer cell group may be calculated from the result obtained from a part of the breast cancer cell group. In addition, the detection step S3 and the counting step S4 may be performed with respect to a plurality of parts of the breast cancer cell group, and an average value of the results thereof may be used as the result of the breast cancer cell group.

The number of drug-sensitive cells or the existence rate thereof can be obtained by steps S1 to S4 described above. Such information, for example, can be used in order to determine the breast cancer cell group sampled from the target or the drug sensitivity of the breast cancer existing in the target.

In one embodiment, step from S1 to S4 may be continuously carried out without performing another step between any of these steps.

As illustrated in FIG. 4, a method according to another embodiment may further include a determination step S5 of determining the presence or absence or the degree of the sensitivity of the breast cancer cell group with respect to the drug from the result of the counting step S4. In this step, the “breast cancer cell group” can be the breast cancer cell group that is used in step S1 and is in a state of being taken out from the body of the target to the outside of the body.

For example, it can be determined that the sensitivity of the breast cancer cell group with respect to the drug is high as the number of drug-sensitive cells in the breast cancer cell group increases. On the contrary, when there is less or no drug-sensitive cells in the breast cancer cell group, it can be determined that the sensitivity of the breast cancer cell group with respect to the drug is low.

In a case where the existence rate of the drug-sensitive cell is calculated, it can be determined that the sensitivity of the breast cancer cell group with respect to the drug is high as the existence rate increases. Alternatively, in a case where the existence rate has a threshold value, and the existence rate is higher than the threshold value, it may be determined that the breast cancer cell group has the sensitivity with respect to the drug. The threshold value is determined in accordance with the type of drug or the type marker gene, and is not limited, but for example, it can be determined that when the existence rate of the drug-sensitive cell is greater than or equal to 10%, the breast cancer cell group has the sensitivity with respect to the drug.

In the determination step S5, the presence or absence or the degree of the drug sensitivity of the breast cancer cell group at a time point when this method is performed or a time point when the breast cancer cell group is sampled may be determined, or the presence or absence or the degree of future drug sensitivity of the breast cancer cell group may be determined. For example, even in a case where a signal that is obtained is faint, but the number of drug-sensitive cells is greater than or equal to the threshold value, it may be determined that there is a possibility of having the drug sensitivity in the future. “Future”, for example, includes the subsequence of the breast cancer existing in the body of the target in which the breast cancer cell group is obtained or the subsequence of the breast cancer cell group maintained in a state suitable to the existence after the method. Herein, “determination” includes the determination of the drug sensitivity of the breast cancer cell group at a time point when this method is performed or a time point when the breast cancer cell group is sampled, and the determination (that is, the prediction) of the future drug sensitivity.

In addition, in the detection step S3, the intensity of the signal may be further measured, and information of the intensity may be used in the determination of the degree of the drug sensitivity of the breast cancer cell group. For example, in a case where the number of drug-sensitive cells is the same, but the intensities of the obtained signal are different, it can be determined that a cell group in which the intensity of the signal is higher has higher drug sensitivity.

According to the method of the embodiment described above, the first nucleic acid is efficiently introduced to the breast cancer cell group by using the lipid particle 1, and the number of drug-sensitive cells (the existence rate) in the living breast cancer cell group is used as an index, and thus, the sensitivity of the breast cancer cell group with respect to the drug can be more accurately determined.

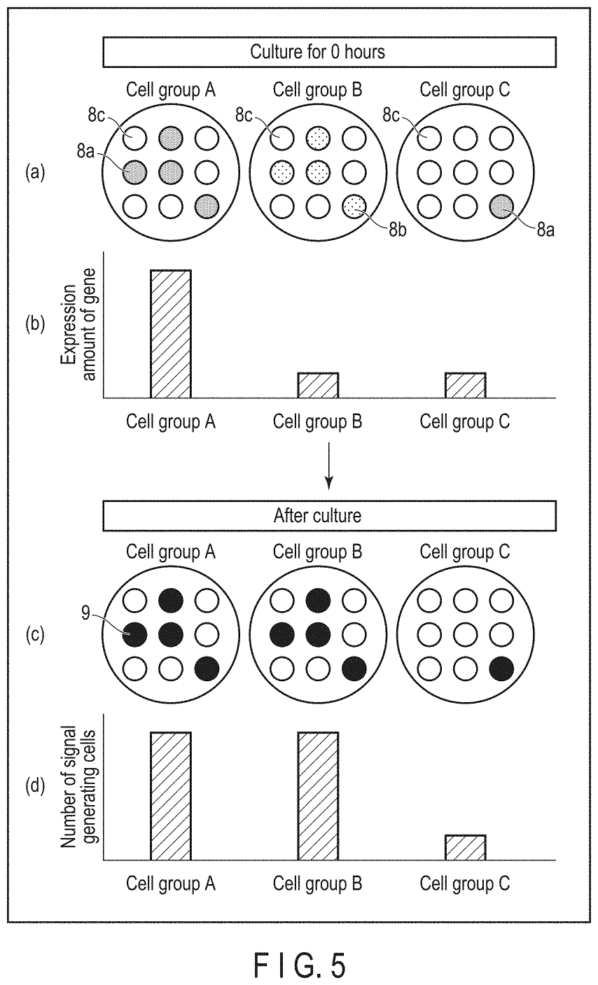

In addition, according to the method of the embodiment, false-negative properties and false-positive properties are prevented, and thus, accurate determination can be performed. An example of the reason will be described by using FIG. 5.

For example, in the case of using three types of cell groups illustrated in part (a) of FIG. 5, that is, a cell group A: including four cells 8a in which the expression amount of the marker gene is high and five cells 8c in which the marker gene is not expressed; a cell group B: including four cells 8b in which the expression amount of the marker gene is low and five cells 8c; and a cell group C: including one cell 8a in which the expression amount of the marker gene is high and nine cells 8c, as illustrated in part (b) of FIG. 5, the expression amount of the marker gene of the entire cell group can be increased in the cell group A, and can be decreased in the cell groups B and C, before the culture (the culture for 0 hours). Accordingly, as with the related art, in the case of using a PCR method or a microarray method in which the culture is not performed and the number of cells is not counted, the cell group B may be determined to be negative even though the number of cells in which the marker gene is expressed is identical to that of the cell group A (the false-negative properties). In addition, in a case where the cell group B is compared with the cell group C, the cell group C has the same result as that of the cell group B in the expression amount of the marker gene even though the number of cells in which the marker gene is expressed is low, and a determination result of the drug sensitivity may be determined to be the same as that of the cell group B (the false-positive properties).

On the other hand, according to the method of the embodiment, the promoter activity of the living breast cancer cell is detected by using the first nucleic acid, and the culture step S2 is further performed, and thus, the reporter protein is continuously expressed during a culture period. The expression of the marker gene can be detected as an enhanced signal after the culture, and thus, as illustrated in part (c) of FIG. 5, the cell 8b in the cell group B can be also detected as the signal generating cell 9, as with the cell 8a. As a result thereof, as illustrated in part (d) of FIG. 5, in the number of signal generating cells, it can be determined that the cell group B has the sensitivity with respect to the drug or has high sensitivity, as with the cell group A, whereas it can be determined that the cell group C including less cells in which the signal is obtained has no drug sensitivity or has low sensitivity.

In a case where there are more cells in which the marker gene is faintly expressed, as with the cell group B, the sensitivity of the entire breast cancer cell group with respect to the drug may be high, compared to a case where there are less cells in which more marker genes are expressed, as with the cell group C. For example, it is also considered that the cell 8b in which the expression amount of the marker gene is low can be the cell 8a in which a future expression amount is high. In addition, for example, the cell 8a in the cell group C is also considered as an outlier and an abnormal value.

Therefore, according to the method of the embodiment, as with the cell group B, at least the breast cancer cell group including more cells in which the marker gene is expressed can be detected to be positive without being false-negative, and as with the cell group C, the breast cancer cell group including less cells in which the marker gene is expressed can be detected to be negative without being false-positive. For this reason, the drug sensitivity can be accurately determined. The number of cells included in the breast cancer cell group or the expression amount of the marker gene is not limited to that illustrated in FIG. 5.

As described above, according to the method of the first embodiment, the false-negative properties and the false-positive properties are prevented, and thus, the drug sensitivity of the breast cancer cell group can be accurately determined.

In one embodiment, step from S1 to S5 may be continuously carried out without performing another step between any of these steps.

According to another embodiment, in a case where a breast cancer cell group derived from the target is used as the breast cancer cell group, the determination result of the drug sensitivity of the breast cancer cell group (the culture cell) obtained by the method of the first embodiment can be used in order to aid the determination of the sensitivity of the breast cancer existing in the body of the target with respect to the drug.

### Second Embodiment

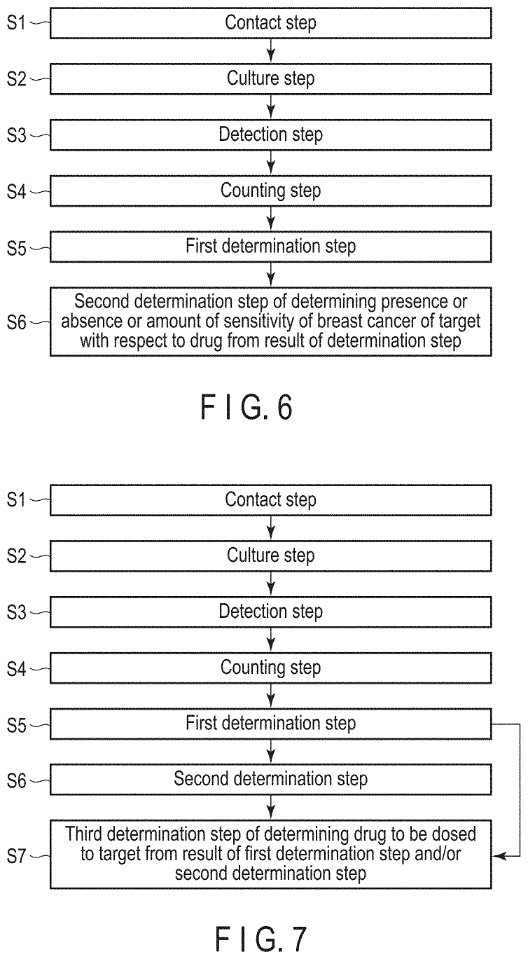

According to a second embodiment, a method of determining, for example, diagnosing the sensitivity of a breast cancer existing in the body of a target with respect to a drug is provided. As illustrated in FIG. 6, this method includes a second determination step S6 of determining the presence or absence or the degree of the sensitivity of the breast cancer existing in the body of the target with respect to the drug from the result of a determination step S5 (here, referred to as a “first determination step S5”), in addition to steps S1 to S5 described above.

A breast cancer cell group that is used in this method is sampled from the target. For example, the result of the breast cancer cell group can be regarded as a representative value of the breast cancer in the body of the target.

Accordingly, for example, in a case where it is determined that the breast cancer cell group has sensitivity with respect to a specific drug or has high sensitivity in the first determination step S5, similarly, it can be determined that the breast cancer in the body of the target has also sensitivity with respect to the drug or has high sensitivity in the second determination step S6. On the contrary, in a case where it is determined that the breast cancer cell group has no sensitivity with respect to a specific drug or has low sensitivity in the first determination step S5, similarly, it can be determined that the breast cancer in the body of the target also has no sensitivity with respect to the drug or has low sensitivity in the second determination step S6.

According to the method of the second embodiment, false-negative properties and false-positive properties are also prevented, and thus, the sensitivity of the breast cancer in the body of the target with respect to the drug can be accurately determined.

In one embodiment, step from S1 to S6 may be continuously carried out without performing another step between any of these steps.

In another embodiment, a drug to be dosed to the target may be selected in accordance with the result of the first determination step S5 and/or the second determination step S6. As illustrated in FIG. 7, for example, this method includes a third determination step S7 of selecting the drug to be dosed to the target after the first determination step S5 or the second determination step S6.

The third determination step S7, for example, includes determining whether or not the drug is dosed to the target, determining whether or not the other drug is dosed to the target, or determining a dose schedule of the drugs, and the like, from the presence or absence of the sensitivity of the breast cancer cell group or the breast cancer of the target with respect to the specific drug or the degree of the sensitivity, which is determined in the first determination step S5 or the second determination step S6.

According to the method of the embodiment, a suitable drug can be more accurately selected. According to this method, the false-negative properties and the false-positive properties can be prevented, and thus, the selection of a drug that is unsuitable to the target is prevented.

In one embodiment, step from S1 to S7 may be continuously carried out without performing another step between any of these steps.

In another embodiment, the second determination step S6 may be performed after the counting step S4 without performing the first determination step S5. That is, the drug sensitivity of the breast cancer in the body of the target may be determined from the number of drug-sensitive cells and the existence rate that are obtained in the counting step S4.

### Third Embodiment

In a third embodiment, a method using a plurality of types of promoter sequences is provided. According to the method of the third embodiment, an expression level can be examined simultaneously with respect to the plurality of types of promoter sequences. For example, (i) promoter sequences of a plurality of sensitivity predictive marker genes respectively corresponding to a plurality of drugs that are different from each other are selected as the plurality of types of promoter sequences, and thus, the sensitivity of a breast cancer cell group with respect to the plurality of drugs can be determined. Alternatively, (ii) promoter sequences of a plurality of types of sensitivity predictive marker genes corresponding to one type of drug are selected as the plurality of types of promoter sequences, and thus, sensitivity with respect to the one type of drug can also be more accurately determined.

A first nucleic acid that is used in the third embodiment includes each promoter sequence of the first to the n-th marker genes for determining the sensitivity of the breast cancer cell with respect to the drug (hereinafter, referred to as the “first to the n-th promoter sequences”), and the first to the n-th reporter genes respectively linked to the downstreams of the first to the n-th promoter sequences to be functionable. The first to the n-th promoter sequences are different from each other, the first to the n-th reporter genes are different from each other, and n is a natural number of greater than or equal to 2.

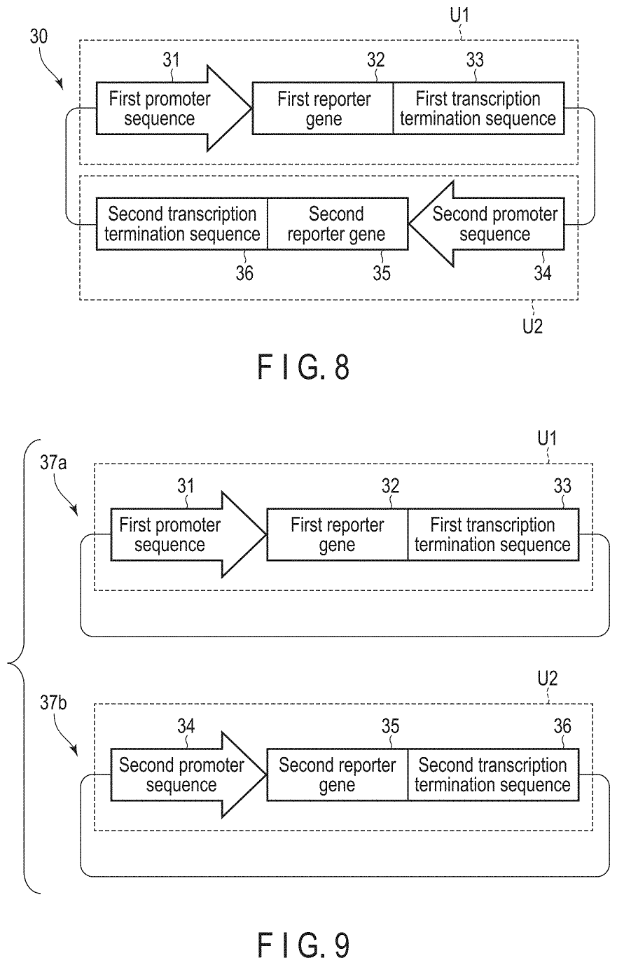

An example of such a first nucleic acid will be described by using FIG. 8. For example, a first nucleic acid 30 includes two reporter gene expression units. A first reporter expression unit U1 includes a first promoter sequence 31, a first reporter gene 32, and a first transcription termination sequence 33. A second reporter expression unit U2 includes a second promoter sequence 34, a second reporter gene 35, and a second transcription termination sequence 36.

The first promoter sequence 31 and the second promoter sequence 34 include promoter sequences of marker genes that are different from each other, respectively. Here, the first promoter sequence 31 and the second promoter sequence 34 may be promoter sequences of marker genes for respectively determining sensitivity with respect to two drugs that are different from each other (corresponding to (i) described above), or may be promoter sequences of two marker genes that are different from each other and are for determining sensitivity with respect to the same drug (corresponding to (ii) described above). For example, the drug and the marker gene can be selected from those described in the first embodiment.

It is preferable that the first reporter gene 32 and the second reporter gene 35 are different types of reporter genes, and signals of reporter proteins that are expressed therefrom are different from each other. Such reporter genes can be selected from those described in the first embodiment. For example, it is preferable to use fluorescent protein genes having fluorescence wavelengths different from each other, luminescent enzyme protein genes having luminescent wavelengths that are emitted from a metabolite and are different from each other, and the like.

The first transcription termination sequence 33 and the second transcription termination sequence 36 may be respectively selected from any of the transcription termination sequences described above, may include sequences that are identical to each other, or may include sequences that are different from each other.

In this example, the type of reporter expression unit included in the first nucleic acid 30 is not limited to two types as illustrated in FIG. 8, and a first nucleic acid including three types or more reporter expression units may be used. In addition, it is not necessary that the directions of each of the reporter expression units (directed toward a 3′ side from a 5′ side or directed toward the 5′ side from the 3′ side) are the same in all of the units, but any of the reporter expression units may be disposed in a different direction.

In another embodiment, the first nucleic acid may include a plurality of sub-nucleic acids respectively including different reporter gene expression units. For example, as illustrated in FIG. 9, a first sub-nucleic acid 37a including the first reporter expression unit U1, and a second sub-nucleic acid 37b including the second reporter expression unit U2 may be used.

The first sub-nucleic acid 37a and the second sub-nucleic acid 37b may be included in the same lipid membrane 2, and may be prepared as one type of lipid particle 1. In this case, each of the sub-nucleic acids may be condensed at once with the nucleic acid condensed peptide and included in the lipid membrane 2, or may be individually condensed and included in the lipid membrane 2. Alternatively, the first sub-nucleic acid 37a and the second sub-nucleic acid 37b may be included in an individual lipid membrane 2 and individually prepared as two types of lipid particles 1.

In this example, the plurality of types of promoter sequences to be used are not limited to two types as illustrated in FIG. 9, and three types or more of promoter sequences may be used.

In addition, in this embodiment, it is not necessary that the nucleic acid is in a circular shape as illustrated in FIG. 8 and FIG. 9, and the nucleic acid may be a linear nucleic acid.

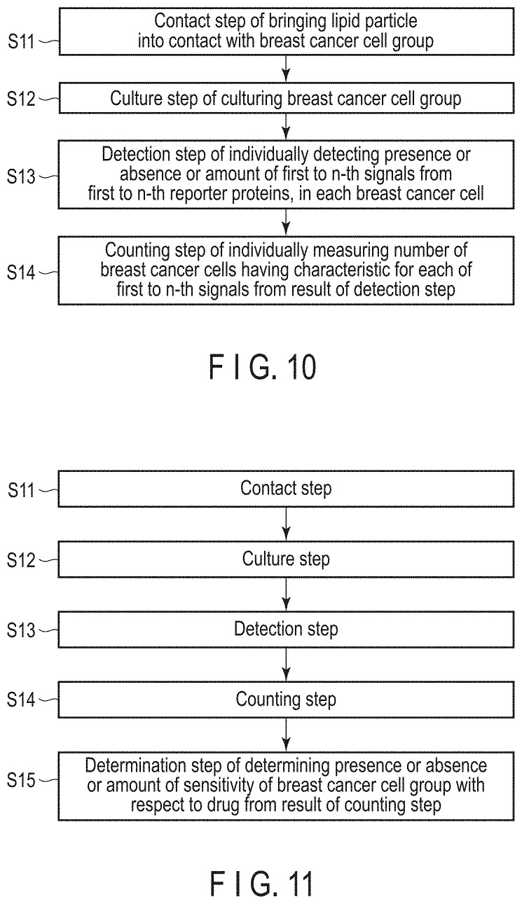

As illustrated in FIG. 10, the method according to the third embodiment, for example, includes the following steps.

(S11) a contact step of bringing a lipid particle including a lipid membrane and a first nucleic acid included in the lipid membrane into contact with a breast cancer cell group;

(S12) a culture step of culturing the breast cancer cell group;

(S13) a detection step of detecting the presence or absence or the amount of each of the first to the n-th signals from the first to the n-th reporter proteins that are respectively expressed from the first to the n-th reporter genes, in each breast cancer cell included in the breast cancer cell group; and

(S14) a counting step of individually measuring the number of breast cancer cells having a characteristic for each of the first to the n-th signals from a result of the detection step.

The contact step S11 can be performed as with the contact step S1 described above except that a first nucleic acid including a plurality of promoter sequences is used as the first nucleic acid. In the case of using a first nucleic acid such as the first sub-nucleic acid 37a and the second sub-nucleic acid 37b included in the individual lipid membrane 2, the lipid particles respectively including the first sub-nucleic acid 37a and the second sub-nucleic acid 37b may be simultaneously or sequentially brought into contact with the breast cancer cell group. At this time, in order to adjust a culture time or to adjust a suitable time for performing the detection step S13, a sub-nucleic acid including a promoter sequence of a marker gene having a weak promoter activity may be introduced early, and a sub-nucleic acid including a promoter sequence of a marker gene having a strong promoter activity may be introduced late.

The culture step S12 can be performed as with the culture step S2 described above.

In the detection step S13, the first to the n-th signals are individually detected from each of the breast cancer cells. Each of the first to the n-th signals is detected by the same method as that of the detection step S3 described above. For example, in a case where the signal is an optical signal, a microscope, an optical sensor, a camera, or the like that is capable of detecting a plurality of light rays having different wavelengths can be used.

In the counting step S14, the number of drug-sensitive cells may be measured for each of the first to the n-th signals by the same method as that of the counting step S4.

For example, it is preferable that each of the signals is detected over time while the culture is performed, and the number of drug-sensitive cells when a signal amount is maximized in each of the signals is measured. As a result thereof, the number of cells can be accurately measured regardless of a difference in an optimal culture time and an optimal detection time according to a difference in the activities of the plurality of promoter sequences.

As illustrated in FIG. 11, the method according to the third embodiment is capable of further including a determination step (S15) of determining the presence or absence or the degree of the sensitivity of the breast cancer cell group with respect to a drug from the result of the counting step. In the determination step S15, (i) in a case where the plurality of promoter sequences respectively correspond to a plurality of types of drugs, as with the determination step S5 described above, the presence or absence or the degree of sensitivity with respect to the plurality of types of drugs is individually determined from the number of drug-sensitive cells that are respectively obtained by the individual detection of the first to the n-th signals, and an existence rate thereof. Accordingly, the sensitivity with respect to the plurality of drugs can be simultaneously determined.

(ii) In a case where the plurality of promoter sequences are a plurality of marker genes corresponding to one type of drug, the sensitivity with respect to the drug may be comprehensively determined from the number of drug-sensitive cells that are respectively obtained by the individual detection of the first to the n-th signals, or the existence rate thereof. For example, the sensitivity may be determined from an average value of the number of drug-sensitive cells in each of the signals by the same method as that of the determination step S5 described above. Accordingly, the sensitivity with respect to one type of drug can be more accurately determined.

According to the steps described above, drug sensitivity of the breast cancer cell group can be accurately determined by using the plurality of promoter sequences.

In one embodiment, step from S11 to S15 may be continuously carried out without performing another step between any of these steps.

In addition, as with the second embodiment (for example, the example illustrated in FIG. 6 and FIG. 7), the method of the third embodiment may further include a second determination step of determining the sensitivity of a breast cancer existing in the body of a target with respect to a drug, and a third determination step of selecting the drug.

### Fourth Embodiment

A method according to a fourth embodiment is a method of determining the characteristic of a tumor cell. That is, in the fourth embodiment, the other tumor cell group may be used instead of the breast cancer cell group in the method according to the first embodiment, and the other characteristic of the tumor cell may be determined instead of the sensitivity with respect to the drug in the method according to the first embodiment.

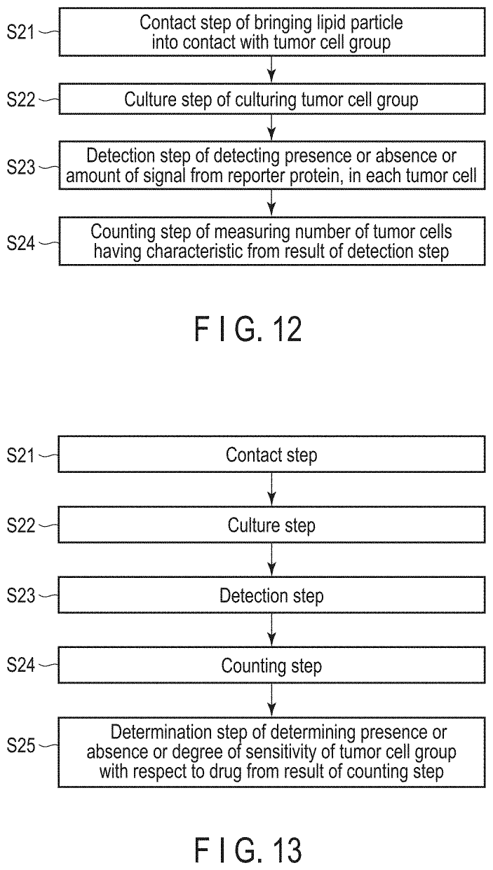

As illustrated in FIG. 12, for example, the method according to the fourth embodiment includes the following steps.

(S21) a contact step of bringing a lipid particle including a lipid membrane and a first nucleic acid included in the lipid membrane into contact with a tumor cell group (the first nucleic acid including a promoter sequence of a marker gene for determining the characteristic of a tumor cell and a reporter gene linked to the downstream of the promoter sequence to be functionable);

(S22) a culture step of culturing the tumor cell group;

(S23) a detection step of detecting the presence or absence or the amount of a signal from a reporter protein that is expressed from the reporter gene, in each tumor cell included in the tumor cell group; and

(S24) a counting step of measuring the number of tumor cells having the characteristic from the result of the detection step.

The other tumor is any malignant tumor that is formed in the body of an animal, and includes a lung cancer, an esophageal cancer, a stomach cancer, a large bowel cancer, a biliary cancer, a pancreatic cancer, a liver cancer, a bladder cancer, an ovary cancer, a prostate cancer, a brain tumor, a sarcoma, a uterine body cancer, a uterus sarcoma, a hematopoietic malignancy, and the like, but is not limited thereto.

The other characteristic, for example, is the prognosis, metastatic properties, infiltrative properties, or the like of the tumor cell. In addition, the characteristic of the tumor cell also includes whether or not a healthy cell becomes a future tumor cell.

In this example, a promoter sequence of a marker gene for determining a desired characteristic in a desired tumor is used as a promoter sequence 5 of a first nucleic acid 3. The promoter sequence can be a promoter sequence of a marker gene relevant to a desired tumor known in the field, for example, a prognosis marker gene, a metastatic marker gene, an infiltrative marker gene, a tumor cell marker gene, or the like.

For example, as a reporter gene 6 and a transcription termination sequence 7, any of those described in the first embodiment can be used.

A material containing the base lipid, the first lipid compound, and the second lipid compound described in the first embodiment and/or the other lipid can be used as the material of a lipid membrane 2.

Steps S21 to S25 of the method according to the fourth embodiment can be performed as with each of steps S1 to S5 described above. Here, a desired characteristic is determined in the determination step S25 by using a desired tumor cell group instead of the breast cancer cell group in the method of the first embodiment, and by using a first nucleic acid including a promoter sequence of a marker gene for determining the other characteristic of the tumor cell instead of the sensitivity with respect to the drug.

As illustrated in FIG. 13, the method according to the fourth embodiment is capable of further including a determination step (S25) of determining the characteristic of the tumor cell group from the result of the counting step. The determination of each characteristic of the tumor cells in the determination step S25 can be performed in accordance with the type of marker gene and the knowledge of a person skilled in the art.

In one embodiment, step from S21 to S25 may be continuously carried out without performing another step between any of these steps.

According to the fourth embodiment, false-negative properties and false-positive properties are prevented, and thus, the characteristic of the tumor cell can be more accurately determined.

In addition, the method of the fourth embodiment may be the same method as that of the second embodiment and may further include a step of determining the characteristic of a tumor existing in the body of the target. In addition, the method of the fourth embodiment may be the same method as that of the third embodiment, and this method may be performed by using a plurality of promoter sequences.

### Fifth Embodiment

(Cell Culture and Detection Device)

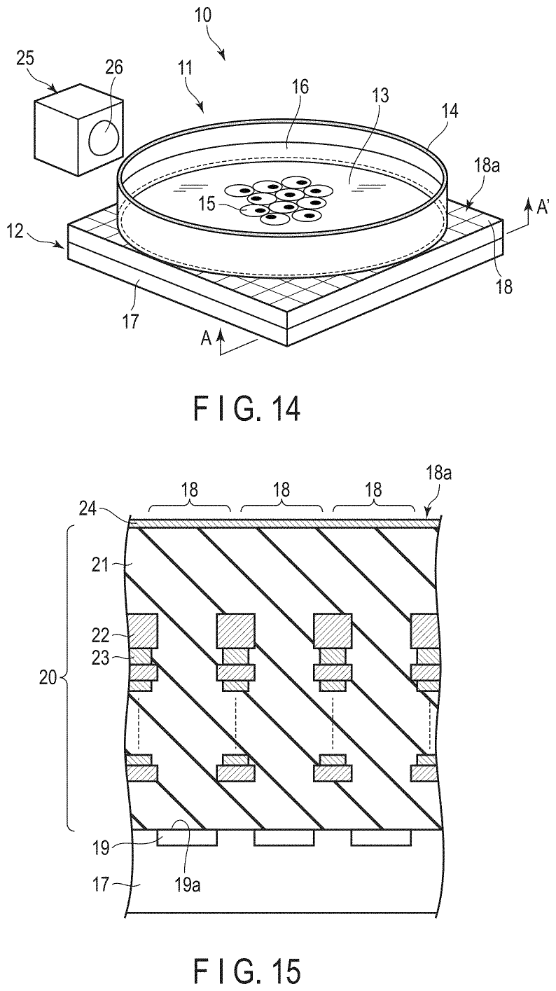

In a fifth embodiment, a cell culture and detection device for performing the culture step, the detection step, and the counting step in the first embodiment to the fourth embodiment described above is provided. As illustrated in FIG. 14, a cell culture and detection device 10, for example, includes a specimen storage portion 11 and an optical sensor 12.

The specimen storage portion 11, for example, includes a bottom portion 13 and a wall portion 14 erected from the circumferential edge of the bottom portion 13, and is in the shape of a liquid-tight container of which the upper portion is opened. The bottom portion 13 and the wall portion 14 contain a light transmissive material. The light transmissive material, for example, can be a material transmitting light such as visible light, an ultraviolet ray, an infrared ray, fluorescence, or chemiluminescence, bioluminescence, or chem-bioluminescence. Examples of such a material include glass, silicon dioxide, polystyrene, polydimethyl siloxane (PDMS), or the like.

Alternatively, for example, commercially available glass, a petri dish, a dish, or a multiwell plate of silicon dioxide, polystyrene, or polydimethyl siloxane (PDMS), and the like may be used as the specimen storage portion 11.

A cell group 15, a culture medium 16, and the like can be contained on the bottom portion 13 of the specimen storage portion 11, and the cell group 15 can be cultured.

The optical sensor 12 includes a plurality of sensor elements 18 arranged into the shape of a matrix on the substrate 17. The plurality of sensor elements 18 are arranged into the shape of a matrix and entirely form a sensor surface 18a that is capable of two-dimensionally detecting optical signal.

FIG. 15 illustrates an enlarged view of three sensor elements 18 on a sectional surface that is cut along A-A′ of FIG. 14. The sensor element 18 includes a sensing unit 19 embedded on the surface of the substrate 17 on the specimen storage portion 11 side. The sensing unit 19, for example, is a light-receiving element that senses light by converting an optical signal into an electrical signal, and any known sensing unit can be used. The sensing unit 19, for example, is a photodiode. A sensing surface 19a receiving the optical signal of the sensing unit 19 is exposed to the surface of the substrate 17.

For example, a wiring layer 20 may be provided on the sensing surface 19a of the sensing unit 19. One layer or a plurality of layers of wires 22 transferring optical information that is sensed by the sensing unit 19 are disposed on the wiring layer 20. The plurality of wires 22, for example, are electrically connected to each other through a via 23 of a conductor material.

The wire 22 and the via 23 are fixed in a desired position by being provided in the light transmissive layer 21. The light transmissive layer 21, for example, contains a light transmissive material such as SiO₂. The wire 22 and the via 23 are provided in a position that does not prevent the light from the specimen storage portion 11 from reaching the sensing unit 19 through the light transmissive layer 21 when the optical sensor 12 is seen in a planar view, for example, between two adjacent sensing units 19.

In addition, the wire 22 and the via 23 function as a light shielding member, prevent light to be detected by the other adjacent sensor element 18 from being penetrated into the sensing unit 19, and thus, improves a detection sensitivity.

A protective layer 24 may be further provided on the surface of the light transmissive layer 21 on a side opposite to the substrate 17. The protective layer 24 is a member for protecting the surface of the light transmissive layer 21, and contains light transmissive material such as a silicon nitride film.

Note that, in FIG. 14, a solid line drawn as a boundary line between the adjacent sensor elements 18 is drawn for convenience in order to represent the sequence of the sensor elements 18, and it may not be necessary that a member for partitioning the sensor element 18 is provided in the portion of such a solid line.

The optical sensor 12, for example, can be formed by a semiconductor process as described below. First, the sensing unit 19 is formed by implanting impurities in a position on the substrate 17 in which the sensing unit 19 is formed. After that, the light transmissive layer 21 is stacked. The wire 22 can be formed while being connected to a desired position in the middle of when the light transmissive layer 21 is stacked through the via 23. The protective layer 24, for example, can be formed by being stacked on the surface of the light transmissive layer 21 by a CVD method.

The area of each of the sensor elements 18 on a surface parallel to the sensor surface 18a, for example, can be 500 nm×500 nm to 10 μm×10 μm. The number of sensor elements 18 included in the optical sensor 12 can be approximately 100 to one hundred million. The number of sensor elements 18 and the size thereof, for example, can be determined in accordance with the size of a cell included in the cell group 15 that is contained in the specimen storage portion 11, a desired definition of an image to be acquired, or the like, but are not limited to the above. It is preferable that the number of sensor elements 18 and the size thereof, for example, are adjusted such that optical information from a cell can be detected by a sensor element 18.

The thickness of the optical sensor 12 can be several μm, for example, 1 μm to 4 μm, but is not limited to such a range.

As the optical sensor 12, a commercially available optical sensor including a plurality of sensor elements that are arranged into the shape of a matrix may be used, and for example, any known CMOS sensor can also be used.

The specimen storage portion 11 is disposed on the sensor surface 18a of the optical sensor 12, and the sensor surface 18a faces the bottom portion 13. A gap may be provided between the sensor surface 18a and the bottom portion 13, and it is preferable that a distance between the sensor surface 18a and the bottom portion 13, for example, is 0 to 100 μm.

In a case where the culture step, the detection step, and the counting step are performed by using this cell culture and detection device 10, first, the culture medium 16, and the cell group 15 in contact with the lipid particle 1 are contained in the specimen storage portion 11. Next, the cell culture and detection device 10 is put in an incubator set to a desired culture condition, or the like, and the cell group 15 is cultured (the culture step).

As illustrated in FIG. 14, the detection step and the counting step, for example, are performed by irradiating the cell group 15 with the light from the light irradiation device 25.

A light source 26 provided in the light irradiation device 25, for example, is a light source applying light such as visible light such as red light, green light, blue light, and white light, ultraviolet light, infrared light, and a combination of two or more selected from the light rays. The light source 26, for example, is an organic EL, LED, a laser, or the like.

The light irradiation device 25 may be fixed to the cell culture and detection device 10, or may be prepared separately without being fixed.

The type of light applied from the light source 26 is selected in accordance with the type of reporter protein. For example, in a case where the reporter protein is a fluorescent protein, the light is excitation light of fluorescence that is generated from itself. In a case where the reporter protein is a luminescent enzyme protein, the light is excitation light of luminescence that is generated from a metabolite of the luminescent enzyme protein. Alternatively, the light may be light for bright-field observation.

As illustrated in FIG. 14, it is preferable that the light, for example, is applied in a direction parallel to the bottom portion 13. As a result thereof, the light is applied to the cell group 15 through the wall portion 14, and is scattered on the cell group 15. In scattered light that is generated, light that is scattered to the lower side is detected by the optical sensor 12. According to such irradiation, light to be a noise is less likely to be generated, and high-sensitive detection can be performed, compared to a case where light is applied from the upper portion or the like of the specimen storage portion 11.

The optical sensor 12 is capable of obtaining the optical information from the cell group 15 contained in the specimen storage portion 11 in relation to two-dimensional position information by the plurality of sensor elements 18. For example, the number of desired cells can be measured from the position and the number of sensor elements 18 in which the signal is obtained. For example, an image or a video may be prepared from a detection result that is obtained from all of the sensor elements 18.

For example, in a case where the optical signal is fluorescence, the detection is performed in a bright-field condition, the total number of cells included in the cell group 15 is measured, and then, the excitation light is applied in a dark-field condition, the optical signal is detected, and the number of signal generating cells is measured. In a case where the reporter gene 6 is a luminescent enzyme protein gene, a step of adding a substrate of an enzyme to the specimen storage portion 11 is further included before the detection step. Alternatively, in a case where the reporter gene 6 is a drug-resistant gene, first, the detection is performed in a bright-field condition, the total number of cells included in the cell group 15 is measured, the corresponding drug is added to the specimen storage portion 11, the detection is performed in a bright-field condition, and the number of remaining cells is measured.

The detection step and the counting step may be performed after the culture, but according to the cell culture and detection device 10, the detection can be simply performed over time while culturing the cell in the specimen storage portion 11.

By using the cell culture and detection device 10 described above, the detection can be simply performed over time during the culture step, and the signal can be detected while maintaining the cell in an environment that is suitable to the existence of the cell, and thus, the characteristic of the tumor cell can be more accurately determined.

In another embodiment, the optical sensor 12 may include a plurality of types of sensor elements 18. The plurality of types of sensor elements 18, for example, are a first sensor element such as the sensor element 18 illustrated in FIG. 15, a second sensor element further including a filter for detecting light at a desired wavelength on the surface or inside, and/or a third sensor element including a filter for detecting light at a wavelength that is different from that of the second sensor element. An optical sensor may be used in which a basic block including a group of sensor elements including each different type of sensor element 18 is arranged into the shape of a matrix in a two-dimensional region.

In addition, in another embodiment, the specimen storage portion 11 may not include the bottom portion 13. In this example, the cell group 15 can be directly contained on the sensor surface 18a of the optical sensor 12, and the cell group 15 can be cultured. Such a cell culture and detection device 10 is capable of including a wall portion 14 erected from the sensor surface 18a.

In this case, the sensor surface 18a and the cell group 15 are in contact with each other, and thus, the optical sensor 12 provided with the basic block described above further including an electrical sensor element detecting an electrical signal derived from the cell group 15, a chemical sensor element detecting a chemical substance or a chemical signal such as pH that is derived from the cell group 15, or the like, in addition to the sensor element detecting the optical signal. By using such an optical sensor 12, for example, the number of signal generating cells in which an electrical signal or a chemical signal from the reporter protein is obtained can be measured.

(Kit)

According to another embodiment, a kit for determining the characteristic of a tumor cell is provided. The kit, for example, includes a cell culture and detection device 10 and a lipid particle 1.

The cell culture and detection device 10 may be any of the above.

The lipid particle 1 is any of the above, and is provided as a reagent for determining the characteristic of the tumor cell. The lipid particle 1, for example, is provided by being contained in a container together with a suitable solvent.

The reagent may further contain a substance for improving storage stability, in addition to the lipid particle 1. The substance for improving the storage stability is not limited, and for example, is glycoprotein such as albumin, lipoprotein, apolipoprotein, and globulin: a pH adjuster, a buffer agent, and a tonicity adjuster; a lipotropic free radical quencher such as α-tocopherol, that suppresses a damage due to a free radical; and a lipid protective agent such as a water-soluble chelator for suppressing an overacidic damage of a lipid and for improving storage stability, such as ferrioxamine.

The reagent may be sterilized by a general method. In addition, the reagent may be provided as the liquid, or may be provided as a powder by being dried. The powdered reagent, for example, can be used by being dissolved in a suitable liquid.

The concentration of the lipid particle 1 contained in the reagent is not limited, and is 0.01 to 30 mass %, and is preferably 0.05 to 10 mass %. The concentration is suitably selected in accordance with the purpose.

The kit may further include a reagent for detecting the signal from the reporter protein. For example, the reagent contains a substrate of a luminescent enzyme protein, a drug corresponding to a drug-resistant gene, or the like.

(Cell Characteristic Determination System)

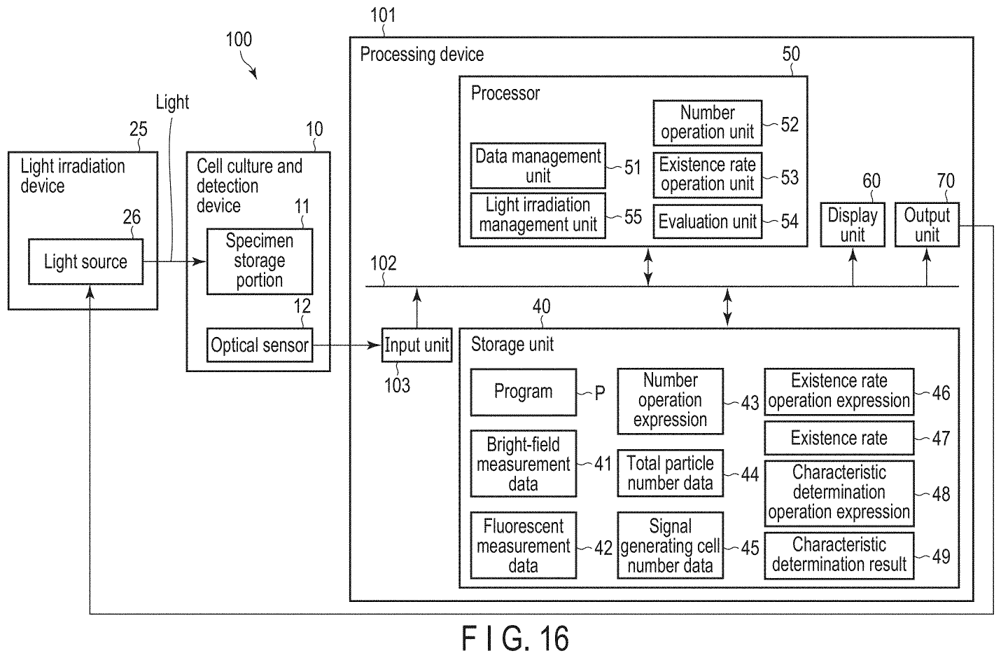

In another embodiment, a cell characteristic determination system is provided. As illustrated in FIG. 16, a cell characteristic determination system 100, for example, includes a cell culture and detection device 10, a light irradiation device 25, and a processing device 101. The processing device 101 includes an input unit 103, a storage unit 40, a processor 50, a display unit 60, and an output unit 70. The input unit 103, the storage unit 40, the processor 50, the display unit 60, and the output unit 70 are electrically connected to each other through a bus 102.

The cell culture and detection device 10 is as described above. The cell culture and detection device 10 can be detachably attached to the cell characteristic determination system 100. In a case where the cell culture and detection device 10 is attached to the cell characteristic determination system 100, an optical sensor 12 is electrically connected to the input unit 103. For example, the cell culture and detection device 10 includes a plurality of pads respectively connected to sensor elements 18 of the optical sensor 12, which is not illustrated, and each of the pads is electrically connected to the input unit 103.

The light irradiation device 25 is as described above. A light source 26 provided in the light irradiation device 25 is disposed to irradiate a specimen storage portion 11 with light.

The input unit 103 is electrically connected to the optical sensor 12, and receives measurement data from the optical sensor 12.

In the storage unit 40, bright-field measurement data 41 and fluorescent measurement data 42 obtained from the optical sensor 12, a number operation expression 43 for measuring the total number of cells and the number of signal generating cells from the bright-field measurement data 41 and the fluorescent measurement data 42, respectively, total cell number data 44, signal generating cell number data 45, an existence rate operation expression 46 for calculating an existence rate of a signal generating cell from each number data item, an existence rate 47 of the signal generating cell, a characteristic determination operation expression 48 for determining a cell characteristic from the existence rate 47, a characteristic determination result 49, and/or a program P are stored.

The program P includes a program for attaining a function of measuring the number of tumor cells having a desired characteristic from the bright-field measurement data 41 and the fluorescent measurement data 42 that are obtained by the optical sensor 12, and of evaluating the characteristic of the tumor cell. The program P may further include a program for controlling the on-off of the light from the light source 26.

The processor 50 includes a data management unit 51, a number operation unit 52, an existence rate operation unit 53, an evaluation unit 54, a light irradiation management unit 55, and the like. The data management unit 51 stores the measurement data that is received through the input unit 103 in the storage unit 40. The number operation unit 52, for example, measures the total number of cells from the bright-field measurement data 41 by using the number operation expression 43, and measures the number of signal generating cells from the fluorescent measurement data 42, based on the program P. The existence rate operation unit 53, for example, calculates the existence rate of the signal generating cell from the total cell number data 44 and the signal generating cell number data 45 by using the existence rate operation expression 46, based on the program P. The evaluation unit 53, for example, determines the cell characteristic from the existence rate 47 by using the characteristic determination operation expression 48, based on the program P. The light irradiation management unit 55 controls the on-off of the light source 26 of the light irradiation device 25 through the output unit 70, based on the program P. The processor 50, for example, is a CPU.

The display unit 60 is capable of including a display, a printer, or the like.

The output unit 70 is electrically connected to the light irradiation device 25, and outputs a command from the light irradiation management unit 55 to the light irradiation device 25.

In addition, the cell characteristic determination system 100 may include a button, a keyboard, a touch panel, or the like for starting or stopping each manipulation of the cell characteristic determination system 100 and for inputting a parameter. The button, the keyboard, the touch panel, or the like, for example, is electrically connected to the input unit 103.

In addition, the cell characteristic determination system 100 may include a culture portion that is not illustrated. The culture portion includes a chamber. In this case, in the chamber, various environment sensors for measuring an environment condition, for example, a temperature, a light condition, a carbon dioxide concentration, an oxygen concentration, and the like, and a facility for adjusting the environment conditions in the chamber, such as a heater, an illumination, and a compressed gas cylinder are provided. The processor 50 is capable of including a condition change unit controlling each of the facilities described above through the output unit such that the environment condition in the chamber is suitable to a culture step, from a measurement result of the environment sensor, based on the program. For example, in a case where the cell culture and detection device 10 is attached into the chamber, the culture step can be performed in the chamber, and the optical sensor 12 can be electrically connected to the input unit 103. In addition, in this case, the light source 26 may be provided in the chamber, and may be disposed to irradiate the specimen storage portion 11 with light from a desired direction.

Next, a method using the cell characteristic determination system 100 will be described.

First, the culture medium 16 and the cell group 15 are contained in the specimen storage portion 11, and the culture step is performed. The cell culture and detection device 10 after the culture step is attached to the cell characteristic determination system 100. In a case where the culture portion is used in the cell characteristic determination system 100, the culture step may be performed by attaching the cell culture and detection device 10 in which the cell group 15 and the culture medium 16 are contained to the culture portion.

Next, the light irradiation management unit 55 commands the light source 26 to be switched on through the output unit 70, and the specimen storage portion 11 is irradiated with light from the light source 26. Then, detection is performed by the optical sensor 12, and the bright-field measurement data 41 and the fluorescent measurement data 42 are obtained. Each of the data items is stored in the storage unit 40 by the data management unit 51 through the input unit 103. At this time, each of the measurement data items in relation to position information of all of the sensor elements 18 is stored in the storage unit 40.

Next, in the number operation unit 52 of the processor 50, the bright-field measurement data 41 and the number operation expression 43 are taken out from the storage unit 40, and the total number of cells is calculated based on the number operation expression 43. The total particle number data 44 is stored in the storage unit 40. In addition, the fluorescent measurement data 42 and the number operation expression 43 are taken out from the storage unit 40, and the number of signal generating cells is calculated based on the number operation expression 43. The signal generating cell number data 45 is stored in the storage unit 40.

Next, in the existence rate operation unit 53 of the processor 50, the total cell number data 44, the signal generating cell number data 45, and the existence rate operation expression 46 are taken out from the storage unit 40, and the existence rate 47 of the signal generating cell is calculated. The existence rate 47 is stored in the storage unit 40. For example, the existence rate 47 may be displayed on the display unit 60.

Next, in the evaluation unit 53 of the processor 50, the existence rate 47 and the characteristic determination operation expression 48 are taken out from the storage unit, and the cell characteristic is determined. The result is stored in the storage unit 40 as the characteristic determination result 49, and is displayed on the display unit 60.

For example, the characteristic determination operation expression may be different in accordance with the type of cell, the type of marker gene, and a cell characteristic to be determined, a plurality of operation expressions corresponding to any combination thereof may be stored in the storage unit 40. For example, the characteristic determination operation expression may be selected based on the type of cell, the type of marker gene, and/or the cell characteristic to be determined, which are input by a manipulator from the input unit, and the cell characteristic may be determined based thereof.

Each procedure described above may be automatically performed by the program P, or may be performed in accordance with the input of the manipulator of this device, or the like.

The execution of the method of this system is ended, and then, the cell culture and detection device 10 can be detached from the cell characteristic determination system 100.

It is not necessary that this system includes the cell culture and detection device 10, and this system may include an attached specimen storage portion and/or an attached optical sensor.

According to the cell characteristic determination system of the embodiment, the characteristic of the tumor cell can be accurately determined.

### EXAMPLES

### Example 1. Preparation of Reporter Vector

A basic vector including an ampicillin-resistant gene sequence, a ColE1 origin sequence, an SV40 origin sequence, a SV40polyA sequence, a NanoLuc gene, and a BGHpolyA sequence was prepared. A restriction enzyme treatment was performed at a SacI site and a XhoI site on the upstream of the NanoLuc gene of the basic vector. After the restriction enzyme treatment, the vector fragment was purified by 0.8% agarose electrophoresis.

A STC2 gene promoter (SEQ ID NOs: 7 and 8 shown in Table 3) and a TOP2A gene promoter (SEQ ID NOs: 9 and 10 shown in Table 3) were amplified by a PCR method (the condition of Table 4) with Human Genomic DNA (manufactured by Novagen Inc.) as a template using a primer set for STC2 gene promoter and a primer set for TOP2A gene promoter respectively. According to the amplification, an STC2 promoter sequence (SEQ ID NO: 1 shown in Table 1) and a TOP2A promoter sequence (SEQ ID NO: 2 shown in Table 2) were respectively obtained. Such amplified products were purified by 0.8% agarose electrophoresis.

TABLE 3

| | | SEQ |
|---|---|---|
| | | ID |
| Primer name | Sequence | NO: |
| Forward primer | TATCGATAGGTACCGAGCT | 7 |
| for STC2 | CTAAAGTTGCCTTTCAAG | |
| | CTCTGGC | |
| Reverse primer | GATCGCAGATCTCGAGTCT | 8 |
| for STC2 | TGGTATTAACCTCCCGTC | |
| | GGG | |
| Forward primer | TATCGATAGGTACCGAGCT | 9 |
| for TOP2A | CTGCAAAGCCTTTCTACA | |
| | TCCTTCC | |
| Reverse primer | GATCGCAGATCTCGAGCTC | 10 |
| for TOP2A | AAGAACCCTGAAAGCGAC | |
| | TAA | |

TABLE 4

| Temperature | Time | | |
|---|---|---|---|
| 98° C. | 3 | min | |
| 98° C. | 10 | sec | 35 |
| 60° C. | 15 | sec | cycle |
| 68° C. | 1 | min | |
| 68° C. | 10 | min | |

| | |
|---|---|
| 4° C. | ∞ |

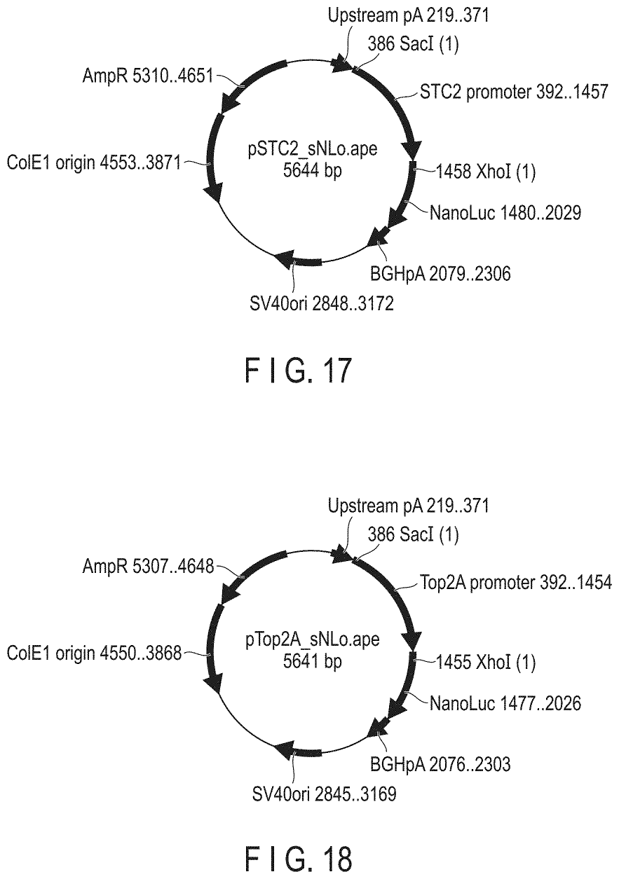

The STC2 gene promoter sequence and TOP2A gene sequence amplified by the PCR method were inserted at the SacI/XhoI sites of the basic vector that was linearized as described above by using InFusion (manufactured by Takara Bio Inc.), and thus, an STC2 reporter vector (FIG. 17) and a TOP2A reporter vector (FIG. 18) were obtained.

### Example 2. Preparation of Lipid Particle Including Reporter Vector

A cationic peptide was added to each of a DNA solution including the STC2 reporter vector and a DNA solution including the TOP2A reporter vector, and thus, a DNA-peptide condensate was formed.

An ethanol-dissolved lipid solution (FFT10/SST04/DOTAP/DOPE/cholesterol/PEG-DMG=37/15/10.5/10.5/30/2 mol) was prepared, and the DNA-peptide condensate described above was added thereto. Further, 10 mM of HEPES (pH 7.3) was gently added, and then, were washed and concentrated by centrifugal ultrafiltration, and thus, a lipid particle including the STC2 reporter vector and a lipid particle including the TOP2A reporter vector were obtained. A DNA inclusion amount of each of the lipid particles was measured by a Quant-iT (Registered Trademark) PicoGreen dsDNA Assay Kit (manufactured by Thermo Fisher Scientific, Inc.).

### Example 3. Introduction to Cell Line of Reporter Vector

Introduction to Cell Line Derived from Human Mammary Gland Tumor

A cell line (MCF-7) derived from a tamoxifen-sensitive human mammary gland tumor and a cell line (MDA-MB-231) derived from a doxorubicin-sensitive human mammary gland tumor were subjected to adhesion culture at 37° C. in 5% CO₂ atmosphere by using a culture medium (MCF-7: MEM (GIBCO), MDA-MB-231: DMEM (GIBCO)) to which 10% fetal bovine serum (FBS) was added in a culture flask.

The culture medium was removed after the culture, and was washed with PBS, and then, the cell was removed by 0.25% Trypsin-EDTA treatment, and then, the cell was suspended in a culture medium to which 10% FBS was added, and Trypsin was inactivated. The cell was collected by centrifugation, and then, the cell was suspended in a culture medium to which 10% FBS was added to be 2.0×10⁵ cells/mL, and each 200 μL of a cell suspension was added to a 96-well culture dish (manufactured by Thermo Fisher Scientific, Inc.) to be 4.0×10⁴ cells/well. Two 96-well culture dishes were prepared, and each of the lipid particles including the reporter vector prepared in Example 2 was added to each of the wells such that the DNA was 0.2 μg/well, and was cultured at 37° C. in 5% CO₂ atmosphere.

Introduction to Human Normal Mammary Gland Epithelial Cell

A human normal mammary gland epithelial cell (HMEC) was subjected to adhesion culture in a culture flask at 37° C. in 5% CO₂ atmosphere by using Mammary Life medium (manufactured by Lifeline Cell Technology LLC.). The culture medium was removed after the culture, and was washed with PBS, and then, the cell was removed by 0.25% Trypsin-EDTA treatment, and then, the cell was suspended in Mammary Life medium, and Trypsin was inactivated. The cell was collected by centrifugation, and then, the cell was suspended in Mammary Life medium to be 2.0×10⁵ cells/mL, and each 200 μL of a cell suspension was added to a 96-well culture dish (manufactured by Thermo Fisher Scientific, Inc.) to be 4.0×10⁴ cells/well. Two 96-well culture dishes were prepared, and each of the lipid particles including the reporter vector prepared in Example 2 was added to each of the wells such that the DNA was 0.2 μg/well, and was cultured at 37° C. in 5% CO₂ atmosphere.

Measurement of NanoLuc Expression Amount (NanoLuc Luciferase Assay)

In 24 hours after a lipid particle including a promoter vector and a breast cancer cell were mixed, a culture plate was taken out from an incubator, a culture medium was removed, and then, the cell was washed with PBS, 100 μL/well of a Glo Lysis Buffer (manufactured by Promega Corporation) was added, and freezing was performed at −80° C. for 30 minutes. Melting was performed at a room temperature, and then, a cell lysate was collected in a centrifugal tube of 1.5 mL. Centrifugal separation was performed at 15000 rpm for 10 minutes and 25 μL of a supernatant was dispensed to a 96-well plate (Black, Nunc). Here, 25 μL of a luciferase substrate solution (Nano Glo Luciferase Assay System, manufactured by Promega Corporation) was added, mixing was performed, and then, a luminescent intensity per one well per 0.1 seconds was measured by using a luminometer (Mithras LB940, manufactured by Berthold Technologies GmbH & Co. KG).

Comparison Result of Promoter Activity

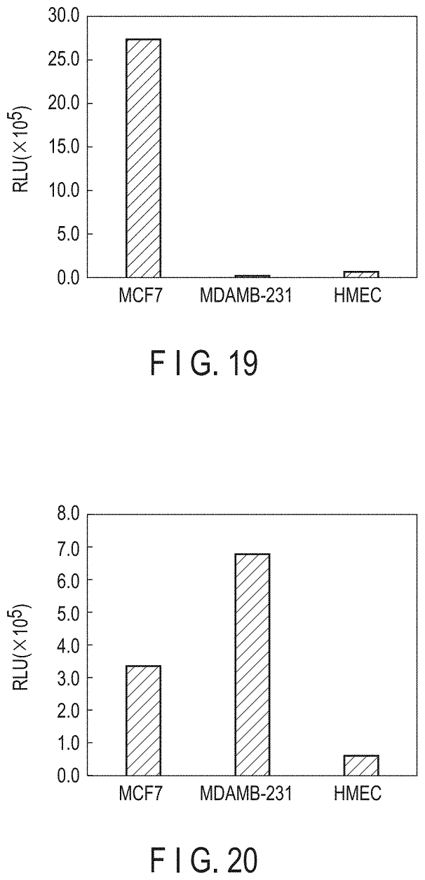

FIG. 19 illustrates a measurement result of a NanoLuc luminescence intensity (RLU, relative light unit) of each breast cancer cell to which a lipid particle including an STC2 promoter vector was introduced. A luminescence intensity of MCF-7 that is a tamoxifen-sensitive breast cancer cell line was approximately 27×10⁵ RLU, which was obviously high, compared to MDA-MB-231 (approximately 0.5×10⁵ RLU) and HMEC (approximately 1×10⁵ RLU). From such a result, it was obvious that the sensitivity of the breast cancer cell with respect to tamoxifen was capable of being determined by the particle including the STC2 promoter vector.

FIG. 20 illustrates a measurement result of a NanoLuc luminescence intensity of each breast cancer cell to which a lipid particle including a TOP2A promoter vector was introduced. A luminescence intensity of MDA-MB-231 that is a doxorubicin-sensitive breast cancer cell line was approximately 7.0×10⁵ RLU, which was obviously high, compared to MCF-7 (approximately 3.5×10⁵ RLU) and HMEC (approximately 0.6×10⁵ RLU). From such a result, it was obvious that the sensitivity of the breast cancer cell with respect to doxorubicin was capable of being determined by the particle including the TOP2A promoter vector.

### Example 4. Introduction of Reporter Vector to Patient Breast Cancer Specimen Cell

Preparation of Specimen Cell and Introduction of Nucleic Acid by Lipid Particle Including DNA

A part of a tumor taken out from a human diseased in a breast cancer by a surgical operation was used as a breast cancer specimen (the specimen was a part of a surgical specimen that was exenterated from a patient with an agreement, based on an experiment plan of a clinical research approved by an ethical committee, and was used in an experiment under suitable management). The specimen was a breast cancer specimen A having sensitivity with respect to tamoxifen, a breast cancer specimen B having sensitivity with respect to doxorubicin, and a specimen C not having sensitivity with respect to both of tamoxifen and doxorubicin. The specimen was sampled, and then, was stored in a sample bottle to which a MACS Tissue Storage Solution (manufactured by Miltenyi Biotec B.V. & CO. KG) was input, and was transported at 4° C. In a cell dispersion of the specimen, MACS Tissue Dissociation Kits (manufactured by Miltenyi Biotec B.V. & CO. KG) were used. 100 μL of an enzyme H (½ amount of a Kit protocol) included with the Kit and 20 μL of an enzyme R (⅕ amount of the Kit protocol) were added to 4.8 ml of RPMI containing 1% BSA, and were put into a gentle MACS C Tube (manufactured by Miltenyi Biotec B.V. & CO. KG) together with the specimen, and the specimen was shredded with scissors. This was set in a gentle MACS-Oct Dissociator with heaters (manufactured by Miltenyi Biotec B.V. & CO. KG), and the cell was dispersed by using a program (Table 5) set for the breast cancer specimen.

TABLE 5

| Number of | |
|---|---|
| rotations | Time |
| +200 rpm | 8″ |
| −200 rpm | 2″ |
| +200 rpm | 2″ |
| −200 rpm | 2″ |
| +200 rpm | 10″ |
| −200 rpm | 2″ |
| +200 rpm | 7″ |
| +20 rpm | 15′ |
| +300 rpm | 13″ |
| −300 rpm | 15″ |
| +500 rpm | 15″ |
| −500 rpm | 15″ |

A specimen cell dispersion liquid was collected in a centrifugal tube through a cell strainer (a nylon filter having a pore size of 70 μm, Product Name: BD Falcon (Registered Trademark)) and was subjected to centrifugation (300 g, 5 minutes, 4° C.). After a supernatant was removed, and a hemolysis treatment was performed in order to remove a red blood cell. The cell was suspended in 5 ml of a hemolysis buffer (obtained by diluting a BD Pharm Lyse Lysing Buffer with sterilized water by 1/10), and the hemolysis treatment was performed at 37° C. for 2 minutes. Next, the centrifugation was performed again (300 g, 5 minutes, 4° C.), and the cell was collected. The precipitate was suspended in Mammary Life medium (manufactured by Lifeline Cell Technology LLC.) and the number of cells was measured using a mix solution of 10 μL cell liquid and 0.4 w/v % of a Trypan Blue Solution (manufactured by Wako Pure Chemical Industries, Ltd., Distribution Source Code: 207-17081) by an automatic cell measure device Countess (Registered Trademark, manufactured by Life Technologies Corporation). Then, cells having a cell diameter of 5 to 60 μm were seeded at the density of 5000 cells/well (15 μL/well).

The NanoCulture dish was used by being coated with 35 mm of a NanoCulture Dish (MS pattern with high adhesiveness)(NCD-HS35-5, manufactured by ORGANOGENIX, Inc.). The coating was performed by placing a gasket having 3 mmΦ and a thickness of 2 mm on the NanoCulture dish, and by adding 15 μL of a solution obtained by diluting iMatrix-511 (manufactured by Nippi, Incorporated, Product Code: 892011) with PBS by 1/60 and a solution obtained by diluting Adhesamine (manufactured by Wako Pure Chemical Industries, Ltd., Distribution Source Code: 010-23201) with PBS by 1/10 to be left to stand at 37° C. for 1 hour. The cell was seeded in each of the wells after a coating solution was removed, and then, the culture was performed at 37° C. in 5% CO₂ condition. Two cell cultures were prepared. In 15 minutes, each of the lipid particles including the promoter vector prepared in Example 2 was added to each of the wells such that the DNA was 0.14 μg/well, and was cultured at 37° C. in 5% CO₂ atmosphere.

Measurement of NanoLuc Expression Amount (NanoLuc Luciferase Assay)

In 24 hours after the lipid particle including the reporter vector was added, the NanoCulture dish was taken out from the incubator, a luciferase substrate (Nano-Glo Luciferase Assay Substrate, manufactured by Promega Corporation) was added to the culture medium to be diluted by 1/1000, the cell was observed by using a luminescent microscope (LuminoView LV200, manufactured by Olympus Corporation) including a high-sensitivity cooling CCD camera (ImageM EX, manufactured by Hamamatsu Photonics K.K.), and a bright-field photograph and a dark-field photograph were captured. Image processing was performed by using MetaMorph (Molecular Device).

Activity Evaluation of STC2/TOP2A Promoters in Specimen Cell

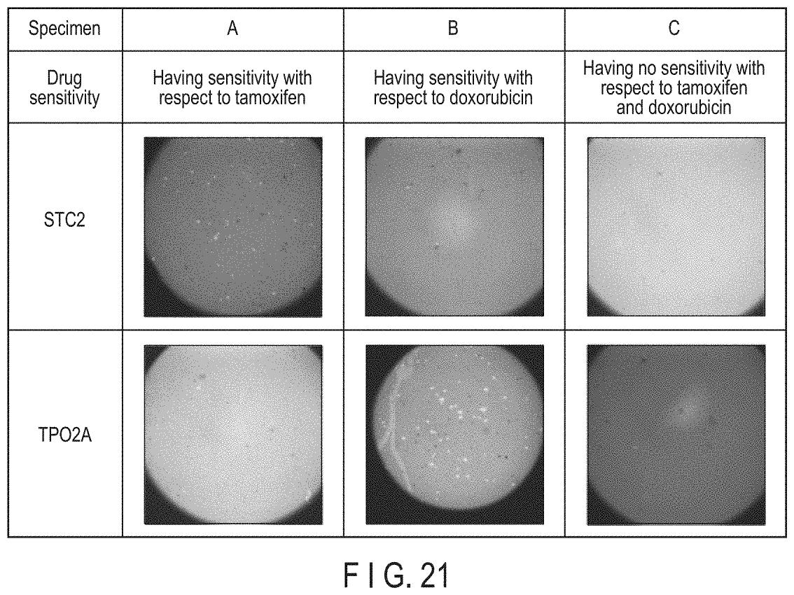

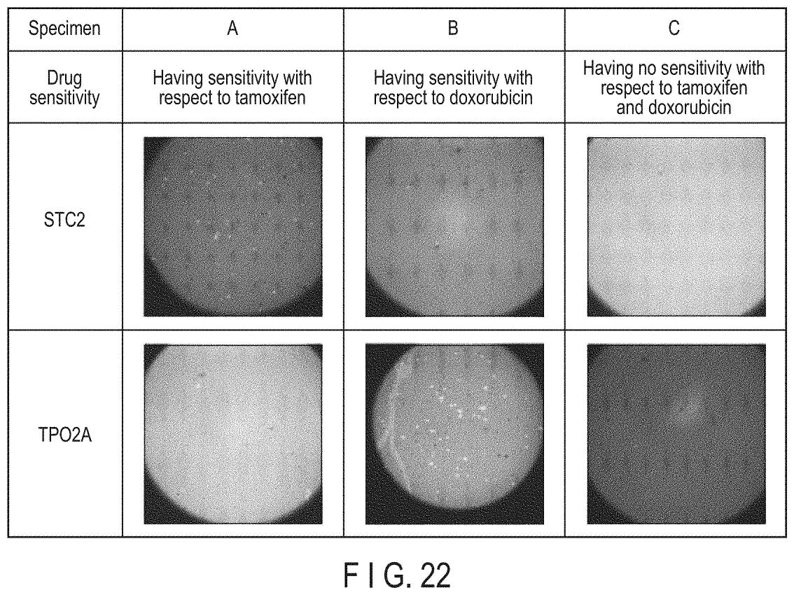

As illustrated in FIG. 21 and FIG. 22, in the photograph of the luminescent cell (NanoLuc-expressing cell) by the lipid particle including the STC2 promoter vector, there were obviously many luminescent cells in the specimen A having sensitivity with respect to tamoxifen, and for example, in a visual field illustrated in FIG. 21 and FIG. 22, approximately 60 luminescent cells were observed with the naked eye. On the other hand, two luminescent cells were observed in the specimen B, and three luminescent cells were observed in the specimen C. So that means those numbers of the cells were obviously smaller than that in the specimen A. FIG. 21 and FIG. 22 are the same photograph in a JPEG format and a bitmap format, respectively.

In the photograph of the luminescent cell (NanoLuc-expressing cell) by the lipid particle including the TOP2A promoter vector, there were obviously many luminescent cells in the specimen B having sensitivity with respect to doxorubicin, and for example, in the visual field illustrated in FIG. 21 and FIG. 22, approximately 70 luminescent cells were observed with the naked eye. On the other hand, two luminescent cells were observed in the specimen A, and four luminescent cells were observed in the specimen C. So that means those numbers of the cells were obviously smaller than that in the specimen A.

From the results described above, it was obvious that the drug sensitivity can be measured in the living breast cancer cell by using the lipid particle including the STC2 promoter vector and the lipid particle including the TOP2A promoter vector.

### Example 5. Comparison with Real-Time PCR Method

Determination of Drug Sensitivity Using Lipid Particle Including Promoter Vector

Breast cancer specimens D and E different from the specimens A, B, and C of Example 4 were used, the lipid particle including the STC2 promoter vector was introduced to the cell by the same method as that of Example 4, and a bright-field photograph and a dark-field photograph were captured. The photograph was subjected to image processing, and thus, the number of cells in which light emission was observed was measured. Image processing was performed by using MetaMorph (Molecular Device). Next, an existence rate of the luminescent cell was calculated as the number of STC2 positive cells/the number of CMV positive cells.

Determination of Drug Sensitivity Using Real-Time PCR Method

RNA was extracted from the cell used in Example 4. The RNA extraction was performed by an RNeasy Mini Kit (Qiagen), and the manipulation was performed in accordance with the manufacture's manual. In a solution of the extracted RNA, a light absorbance at 260 nm was measured by a spectrophotometer, and the amount of RNA was calculated. Then, reverse transcription PCR was performed by using a First-Strand cDNA synthesis kit (manufactured by GE Healthcare Inc.). The manipulation was performed in accordance with the manufacture's manual. cDNA was synthesized by preparing the total RNA of each sample to be 100 ng.

From the cDNA, the expression of STC2 was detected with a StepOnePlus device (manufactured by Applied Biosystems, LLC) by using TaqMan Gene Expression Assay (manufactured by Applied Biosystems, LLC) and TaqMan probe Hs01063215_m1. In an amplification condition of PCR, 50° C. for 2 minutes, 95° C. for 10 minutes, and then, 95° C. for 15 seconds, and 60° C. for 1 minute were set to one cycle, measurement was performed up to 22 cycles, and the presence or absence of amplification was measured by a turbidity.

Comparison of Results

Table 6 shows the existence rate of the luminescent cell in the method using the lipid particle including the promoter vector and the presence or absence of an amplified product of STC2 in the real-time PCR method.

TABLE 6

| | Presence or absence of | Positive cell rate |
|---|---|---|
| | amplification | by lipid particle |
| | by real-time PCR | including promoter vector |
| Test body D | x | 6.50% |
| Test body E | ∘ | 5.70% |

In the method using the lipid particle including the promoter vector, the expression of the STC2 gene was capable of being detected in both of the specimen D and the specimen E, and in the PCR method, the amplified product of STC2 was detected in the specimen E, but the expression was not detected in the specimen D.

From the results described above, it was indicated that in the method using the lipid particle including the promoter vector according to the embodiment, information with respect to the drug sensitivity of the breast cancer cell was accurately obtained, compared to the case of using the PCR method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method for determining a characteristic of a tumor cell group, the method comprising:
bringing a lipid particle including a lipid membrane and a first nucleic acid included in the lipid membrane into contact with the tumor cell group,
wherein the first nucleic acid includes a promoter sequence of a marker gene for determining the characteristic and a reporter gene linked to a downstream of the promoter sequence to be functionable,
culturing the tumor cell group;
detecting the presence or absence or an amount of a signal from a reporter protein that is expressed from the reporter gene, in each tumor cell included in the tumor cell group; and
counting the number of tumor cells having the characteristic that is measured from a result of the detecting.

2. The method according to claim 1, further comprising first determining the presence or absence or a degree of the characteristic of the tumor cell group from a result of the counting.

3. The method according to claim 1, wherein
the tumor cell group is sampled from a tumor in a body of a target, and
the method further comprises: second determining the presence or absence or a degree of the characteristic of the tumor in the body of the target from a result of the counting.

4. The method according to claim 2, wherein
the tumor cell group is sampled from a tumor in a body of a target, and
the method further comprises: second determining the presence or absence or the degree of the characteristic of the tumor in the body of the target from a result of the first determining.

5. The method according to claim 4, further comprising third determining a drug for being dosed to the target that is selected based on the result of the first determining and/or a result of the second determining.

6. The method according to claim 2, wherein
the counting further includes measuring the total number of tumor cells included in the tumor cell group, and
the first determining includes calculating an existence rate of the tumor cell having the characteristic from the total number of tumor cells and the number of tumor cells having the characteristic, and determining the presence or absence or the degree of the characteristic of the tumor cell group from the existence rate.

7. The method according to claim 1, wherein
the first nucleic acid includes each promoter sequence of first to n-th marker genes for determining the characteristic (hereinafter, referred to as “first to n-th promoter sequences”), and first to n-th reporter genes respectively linked to downstreams of the first to the n-th promoter sequences to be functionable,
the first to the n-th promoter sequences are different from each other,
the first to the n-th reporter genes are different from each other, and n is a natural number of greater than or equal to 2,
the detecting includes detecting each of first to n-th signals from first to n-th reporter proteins that are respectively expressed from the first to the n-th reporter genes, and
the counting includes individually measuring the number of tumor cells having the characteristic for each of the first to the n-th signals.

8. The method according to claim 1, wherein the characteristic is sensitivity with respect to a drug, prognosis, metastatic properties, or infiltrative properties.

9. The method according to claim 8, wherein the tumor cell is a breast cancer cell, and the characteristic is the sensitivity with respect to the drug.

10. The method according to claim 9, wherein the drug is an anthracycline-based drug, an antimicrotubule drug, an alkylating compound, an antimetabolic drug, a platinum complex, an aromatase inhibitor, an LH-RH agonist formulation, an anti-estrogen drug, a progestational agent, or a molecularly targeted drug.

11. The method according to claim 10, wherein the drug is the anthracycline-based drug, and the marker gene is TOP2A, RARA, CDCl₆, THRA, GSDM1, PSMD3, CSF3, MED24, SNORD124, NR1D1, TRNASTOP-UCA, MSL-1, CASC3, RAPGEFL1, WIPF2, LOC100131821, GJD3, LOC390791, LOC728207, IGFBP4, TNS4, CCR7, or SMARCE1.

12. The method according to claim 10, wherein the drug is the aromatase inhibitor, and the marker gene is STC2, SLC39A6, CA12, ESR1, PDZK1, NPY1R, CD2, MAPT, QDPR, AZGP1, ABAT, ADCY1, CD3D, NAT1, MRPS30, DNAJC12, SCUBE2, KCNE4, DHA, ATP5J2, VDAC2, DARS, UCP2, UBE2Z, AK2, WIPF2, APPBP2, or TRIM2.

13. The method according to claim 9, wherein the lipid membrane contains at least one component selected from the group consisting of:

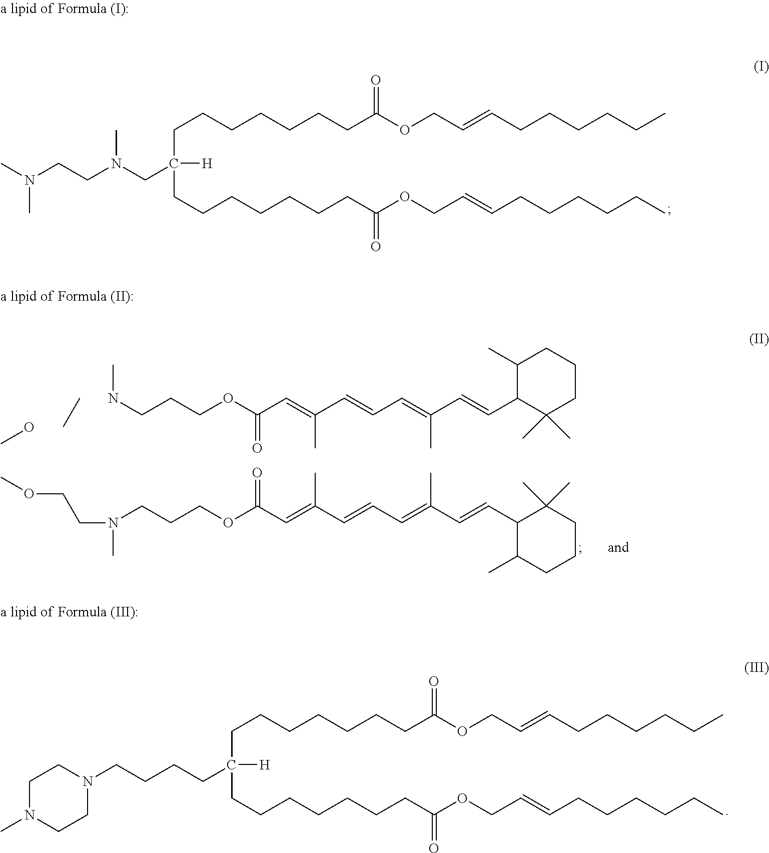

14. The method according to claim 1, wherein in the detecting, the signal is detected over time.

15. The method according to claim 1, wherein
at least the detecting is performed by using a cell culture and detection device, and
the cell culture and detection device comprises a plurality of sensor elements arranged into the shape of a matrix in a two-dimensional region, and a specimen storage portion including a light transmissive bottom portion facing the sensor elements.

16. A kit for determining a characteristic of a tumor cell group, the kit comprising:
a lipid particle including a lipid membrane and a first nucleic acid included in the lipid membrane (the first nucleic acid including a promoter sequence of a marker gene for determining the characteristic and a reporter gene linked to a downstream of the promoter sequence to be functionable); and
a cell culture and detection device comprising a plurality of sensor elements arranged into the shape of a matrix in a two-dimensional region, and a specimen storage portion including a light transmissive bottom portion facing the sensor elements.

17. The kit according to claim 16, further comprising a reagent for detecting a signal from a reporter protein that is expressed from the reporter gene.

18. The kit according to claim 16, wherein
the first nucleic acid includes each promoter sequence of first to n-th marker genes for determining the characteristic (hereinafter, referred to as “first to n-th promoter sequences”), and first to n-th reporter genes respectively linked to downstreams of the first to the n-th promoter sequences to be functionable,
the first to the n-th promoter sequences are different from each other,
the first to the n-th reporter genes are different from each other, and
n is a natural number of greater than or equal to 2.

19. The kit according to claim 16, wherein the characteristic is sensitivity with respect to a drug, prognosis, metastatic properties, or infiltrative properties.

20. The kit according to claim 19, wherein the tumor cell is a breast cancer cell, and the characteristic is the sensitivity with respect to the drug.

21. The kit according to claim 20, wherein the drug is an anthracycline-based drug, an antimicrotubule drug, an alkylating compound, an antimetabolic drug, a platinum complex, an aromatase inhibitor, an LH-RH agonist formulation, an anti-estrogen drug, a progestational agent, or a molecularly targeted drug.

22. The kit according to claim 21, wherein the drug is the anthracycline-based drug, and the marker gene is TOP2A, RARA, CDCl₆, THRA, GSDM1, PSMD3, CSF3, MED24, SNORD124, NR1D1, TRNASTOP-UCA, MSL-1, CASC3, RAPGEFL1, WIPF2, LOC100131821, GJD3, LOC390791, LOC728207, IGFBP4, TNS4, CCR7, or SMARCE1.

23. The kit according to claim 21, wherein the drug is the aromatase inhibitor, and the marker gene is STC2, SLC39A6, CA12, ESR1, PDZK1, NPY1R, CD2, MAPT, QDPR, AZGP1, ABAT, ADCY1, CD3D, NAT1, MRPS30, DNAJC12, SCUBE2, KCNE4, DHA, ATP5J2, VDAC2, DARS, UCP2, UBE2Z, AK2, WIPF2, APPBP2, or TRIM2.

24. The kit according to claim 20, wherein the lipid membrane contains at least one component selected from the group consisting of:

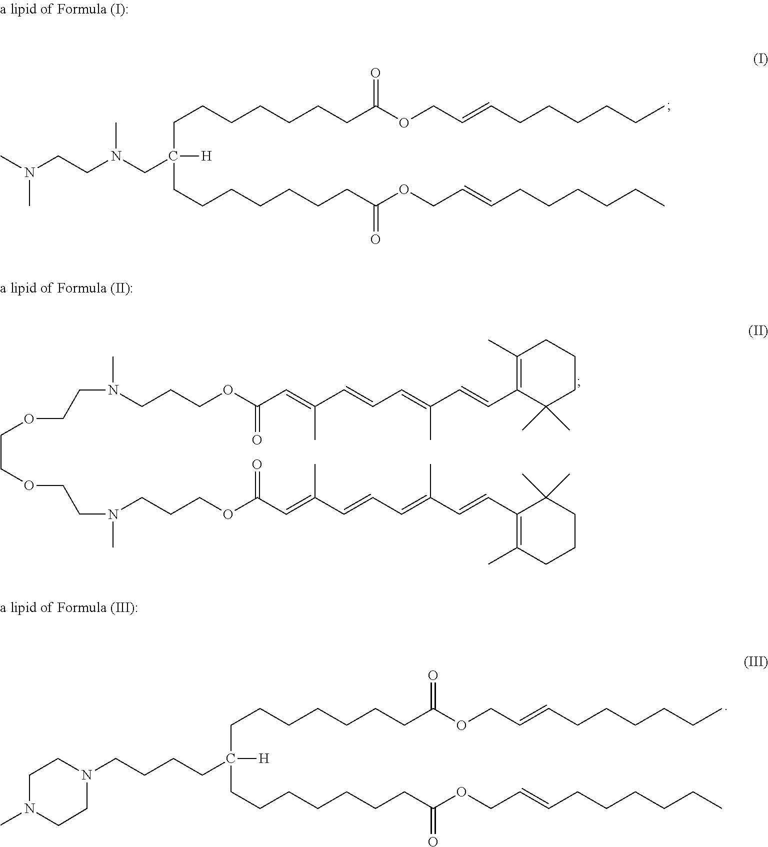

25. A program for attaining a function of counting a tumor cell having a characteristic from the presence or absence or an amount of a signal from a reporter protein that is expressed from a reporter gene of each tumor cell included in a tumor cell group, which is obtained by using the kit according to claim 16, and of determining the characteristic of the tumor cell from a result of the counting.

26. The program according to claim 25, wherein the tumor cell is a breast cancer cell, and the characteristic is sensitivity with respect to a drug.
